(19) United States
(12) Patent Application Publication
(10) Pub. No.: US 2024/0166603 A1
(43) Pub. Date: May 23, 2024

(54) Method of Treating Cancer With Atpenin A5 Derviatives

(71) Applicants: Texas Tech University System, Lubbock, TX (US); University of Rochester Medical Center, Rochester, NY (US)

(72) Inventors: Paul Trippier, Papillion, NE (US); Paul Brookes, Rochester, NY (US); Xiaoyu Hao, Rochester, NY (US)

(21) Appl. No.: 18/322,187

(22) Filed: May 23, 2023

Related U.S. Application Data
(63) Continuation-in-part of application No. 17/054,235, filed on Nov. 10, 2020, now abandoned.
(60) Provisional application No. 62/670,479, filed on May 11, 2018.

(51) Int. Cl. C07D 213/69; A61P 35/00; C07D 213/74; C07D 491/056; C07F 9/58

(52) U.S. Cl. CPC C07D 213/69; A61P 35/00; C07D 213/74; C07D 491/056; C07F 9/58

(57) ABSTRACT

The present invention includes molecules, composition, and methods for making and using a molecule having the formula:

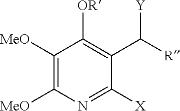

wherein R′ is selected from H, methoxy, methoxymethyl, glycosyl, phosphoryl, alkylamine, polyethylene glycol (PEG), or deuterated; X is selected from OH, methoxy, methoxymethyl, O-methoxymethyl, carbonyl, or alcohol; Y is O; and R″ is selected from H, butyl, pentyl, hexyl, heptyl, octyl, nonyl, decyl, or dodecyl, that are saturated or unsaturated, PEG, or triphenylphosphate groups; optionally one or both MeO groups are deuterated; and N is N-oxide or N-alkyl.

### CROSS-REFERENCE TO RELATED APPLICATIONS

This application claims priority to and is a continuation-in-part of U.S. application Ser. No. 17/054,235, filed Nov. 10, 2020, which is the National Stage of International Application No. PCT/US2019/031444, filed on May 9, 2019 and claims priority to U.S. Provisional Application Ser. No. 62/670,479, filed May 11, 2018, the entire contents of each of which are incorporated herein by reference.

### STATEMENT OF FEDERALLY FUNDED RESEARCH

This invention was made with government support under R01-HL071158 awarded by the National Institutes of Health. The government has certain rights in the invention.

### TECHNICAL FIELD OF THE INVENTION

The present invention relates in general to the field of novel compositions and methods for the treatment of cancer, and more particularly, novel Atpenin A5 derivatives for the treatment of, e.g., prostate cancer and chronic obstructive pulmonary disease (COPD).

### BACKGROUND OF THE INVENTION

Without limiting the scope of the invention, its background is described in connection with treatments for prostate cancer.

Mitochondrial respiratory complex II (CII), also known as succinate dehydrogenase (SDH) or succinate-coenzyme Q reductase (SQR), is a 124 kDa protein complex located in the inner membrane of mitochondria. ^{[1]} CII forms part of the electron transport chain (ETC) as well as being implicated in succinate signaling and substantial reactive oxygen species (ROS) generation.^{[2]} The protein connects the tricarboxylic acid cycle (TCA) and the ETC, while lacking any contribution to maintaining the proton gradient across the mitochondrial inner membrane.^{[3]}

Despite the association of some aggressive forms of cancer with CII mutation (probably linked to hypoxia-inducible factor (HIF) activation resulting from inhibition of HIF prolyl-hydroxylases by succinate accumulation),^{[4]} far greater evidence supports an anti-proliferative role for CII inhibition. Five facets of CII inhibition, beyond the generation of ROS,^{[5]} have significant promise for the development of selective small molecule chemotherapeutics. i) CII is rarely mutated within cancers and thus presents a unique invariant target to address cancer chemotherapy. Mutations that are observed are only associated in infrequent and nonaggressive neoplasias such as pheochromocytomas.^{[6]} In most common cancers such as prostate cancer, only one in a million patients carry mutation in CII.^{[3]} ii) Inhibition of the TCA cycle impairs glutaminolysis, a major source of energy for tumor cells. iii) Along with complex I, complex II (also known as fumarate reductase) is a key member of the NADH-fumarate reductase system, which is important for maintaining mitochondrial energy production in tumor microenvironments under hypoxic conditions.^{[7]} iv) CII inhibition leads to prolonged activation of autophagy and cancer cell death.^{[8]}As cancer cells are known to develop resistance to apoptosis,^{[9]} activation of autophagy could be a viable strategy to combat drug resistance. v) Promisingly, low potency CII inhibitors are selectively cytotoxic to cancer cells, albeit with weak effect, while conveying minimal, to no toxicity, to non-malignant cells.^{[10]}

Several CII inhibitors are known, but all suffer from low potency. 3 -Bromopyruvate (1, 3BP, FIG. 1) was the first identified CII inhibitor, however no IC₅₀ has been reported. Malonate (2) possesses an IC₅₀=40 μM.^{[11]} The vitamin E analogue α-Tocopheryl succinate (α-TOS, 3) has a CII IC₅₀=42 μM,^{[12]} inducing apoptosis in cancer cells by ROS generation. Mitochondrially targeted vitamin E succinate (MitoVES, 4) has an IC₅₀=70 μM.^{[13]} However, MitoVES was found to be 20-50 times more effective in inducing apoptosis in cancer cells than α-TOS.^{[14]} This was attributed to the introduction of a cationic triphenylphosphonium (TPP) group which acts to target the compound to the mitochondria. Inhibition of CII was selective to cancer cells with MitoVES possessing an IC50 of 0.5-3 μM for apoptosis induction in cancer cells and approximately 20-60 μM for non-malignant cells.^{[14]} Thenoyltrifluoroacetone (TFFA, 5), is widely used as a control compound in CII assay kits with an IC₅₀=30 μM.^{[15]} The clinical vasodilator and CII inhibitor diazoxide 6 (IC₅₀=32 μM),^{[16]} is known to regulate ROS production inducing specific cancer cell death.^{[17]}

Atpenin A5 is a potent inhibitor of succinate-coenzyme Q reductase (SQR) also known as mitochondrial respiratory complex II. SQR plays a major role in both the citric acid cycle and the electron transport chain and is found in the inner mitochondrial membrane of eukaryotic cells.

Atpenin A5 has been shown to inhibit the growth of cancer cells in vitro but this success does not translate to in vivo studies. Therefore, a need remains for a potent inhibitor of SQR that is safe and effective for the treatment of cancers.

### SUMMARY OF THE INVENTION

As embodied and broadly described herein, an aspect of the present disclosure relates to a

compound comprising: an Atpenin 5 derivative of Formula:

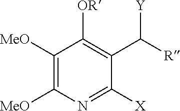

wherein R′ is selected from H, methoxy, methoxymethyl, glycosyl, phosphoryl, alkylamine, polyethylene glycol (PEG), or deuterated; X is selected from OH, methoxy, methoxymethyl, O-methoxymethyl, carbonyl, or alcohol; Y is O; R″ is selected from H, pentyl, hexyl, heptyl, octyl, nonyl, decyl, or dodecyl, that are saturated or unsaturated, a PEG, a triphenylphosphate group; optionally one or both MeO groups are deuterated; and N is N-oxide or N-alkyl. In one aspect, the compound comprises:

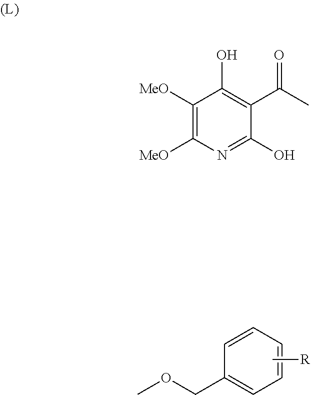

L is a linker that is C₁-C₁₅ or polyethyleneglycol 1-7 units, wherein L can be odd or even; R=2,3 or 4 mono, di, tri substitution with halogen electron-donating group or electron-withdrawing groups; and Ether Oxygen may be NH, S, or C. In another aspect, the compound comprises:

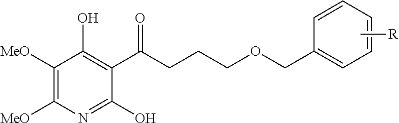

R=2,3 or 4 mono, di, tri substitution with halogen electron-donating group or electron-withdrawing groups; and Ether Oxygen may be NH, S, or C. In another aspect, the compound comprises:

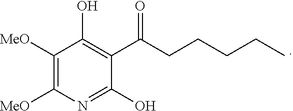 [SMILES: CCCCCC(=O)c1c(O)nc(OC)c(OC)c1O]

In another aspect, the compound comprises:

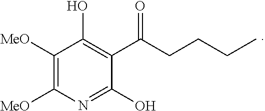 [SMILES: CCCCC(=O)c1c(O)nc(OC)c(OC)c1O]

In another aspect, the compound comprises:

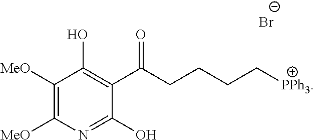

In another aspect, the compound comprises:

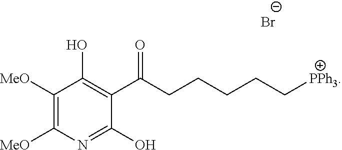

In another aspect, the compound comprises at least one of:

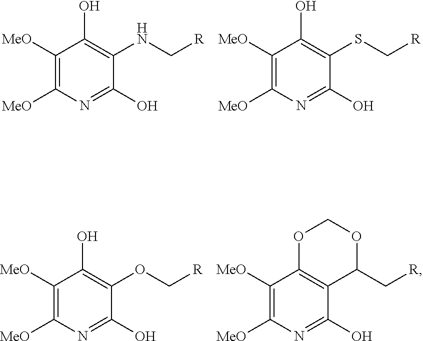

wherein NH is optionally alkylated, sulphur oxidation state will vary S(O) or S(O₂), and R is R′ or R″. In another aspect, the compound comprises:

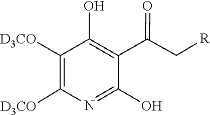

wherein NH is optionally alkylated, sulphur oxidation state will vary S(O) or S(O₂), and R is R′ or R″. In another aspect, the compound is adapted for oral, intravenous, subcutaneous, parenteral, enteral, transcutaneous, transdermal, or rectal administration. In another aspect, the compound further comprises one or more pharmaceutically acceptable carriers, excipients, buffers, or salts.

As embodied and broadly described herein, an aspect of the present disclosure relates to a composition comprising a compound of Formula:

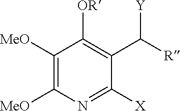

wherein R′ is selected from H, methoxy, methoxymethyl, glycosyl, phosphoryl, alkylamine, polyethylene glycol (PEG), or deuterated; X is selected from OH, methoxy, methoxymethyl, O-methoxymethyl, carbonyl, or alcohol; Y is O; R″ is selected from H, pentyl, hexyl, heptyl, octyl, nonyl, decyl, or dodecyl, that are saturated or unsaturated, PEG, or triphenylphosphate group; optionally one or both MeO groups are deuterated; and N is N-oxide or N-alkyl; and one or more pharmaceutically acceptable carriers. In one aspect, the compound comprises:

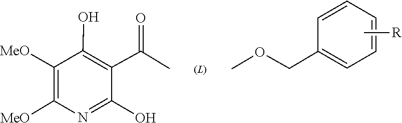

L is a linker that is C₁-C₁₅ or polyethyleneglycol 1-7 units, wherein L can be odd or even; R=2,3 or 4 mono, di, tri substitution with halogen electron-donating group or electron-withdrawing groups; and Ether Oxygen may be NH, S, or C. In another aspect, the compound comprises:

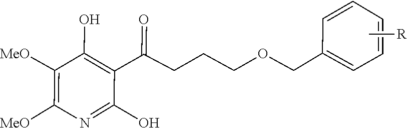

R=2,3 or 4 mono, di, tri substitution with halogen electron-donating group or electron-withdrawing groups; and Ether Oxygen may be NH, S, or C. In another aspect, the compound comprises:

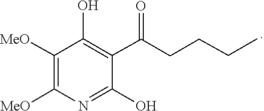 [SMILES: CCCCC(=O)c1c(O)nc(OC)c(OC)c1O]

In another aspect, the compound comprises:

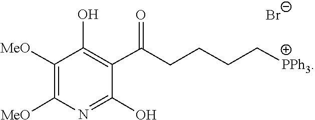

In another aspect, the compound comprises:

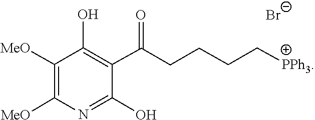

In another aspect, the compound comprises at least one of:

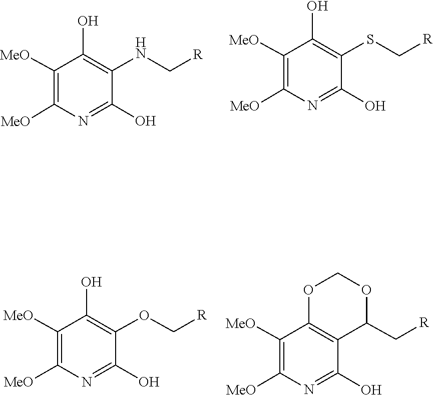

wherein NH is optionally alkylated, sulphur oxidation state will vary S(O) or S(O₂), and R is R′ or R″. In another aspect, the compound comprises:

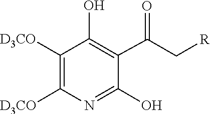

wherein NH is optionally alkylated, sulphur oxidation state will vary S(O) or S(O₂), and R is R′ or R″. In another aspect, the composition is adapted for oral, intravenous, subcutaneous, parenteral, enteral, transcutaneous, transdermal, or rectal administration. In another aspect, the one or more pharmaceutically acceptable carriers is selected from one or more excipients, buffers, or salts to the composition.

As embodied and broadly described herein, an aspect of the present disclosure relates to a method of treating a cancer cell comprising: providing a subject in need of treating the cancer cell with an effective amount of a composition comprising an Atpenin 5 derivative of Formula:

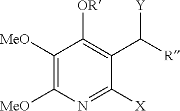

wherein R′ is selected from H, methoxy, methoxymethyl, glycosyl, phosphoryl, alkylamine, polyethylene glycol (PEG), or deuterated; X is selected from OH, methoxy, methoxymethyl, O-methoxymethyl, carbonyl, or alcohol; Y is O; R″ is selected from H, pentyl, hexyl, heptyl, octyl, nonyl, decyl, or dodecyl, that are saturated or unsaturated, PEG or triphenylphosphate group; optionally one or both MeO groups are deuterated; N is N-oxide or N-alkyl; and one or more pharmaceutically acceptable carriers, excipients, buffers, or salts. In one aspect, the compound comprises:

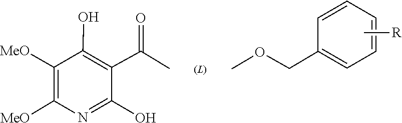

L is a linker that is C₁-C₁₅ or polyethyleneglycol 1-7 units, wherein L can be odd or even; R=2,3 or 4 mono, di, tri substitution with halogen electron-donating group or electron-withdrawing groups; and Ether Oxygen may be NH, S, or C. In another aspect, the compound comprises:

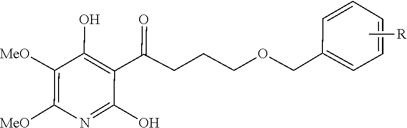

R=2,3 or 4 mono, di, tri substitution with halogen electron-donating group or electron-withdrawing groups; and Ether Oxygen may be NH, S, or C. In another aspect, the compound comprises:

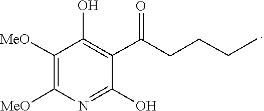 [SMILES: CCCCC(=O)c1c(O)nc(OC)c(OC)c1O]

In another aspect, the compound comprises:

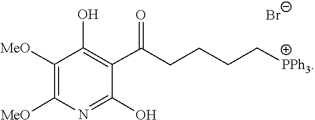

In another aspect, the compound comprises:

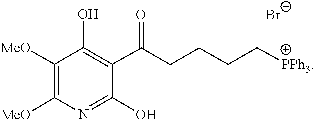

In another aspect, the compound comprises at least one of:

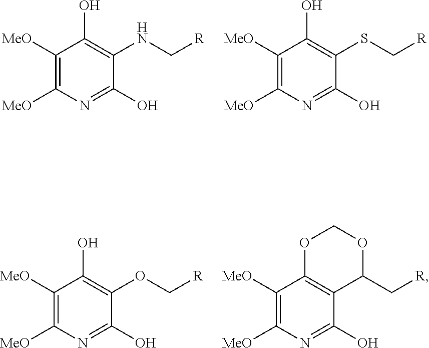

wherein NH is optionally alkylated, sulphur oxidation state will vary S(O) or S(O₂), and R is R′ or R″. In another aspect, the compound comprises:

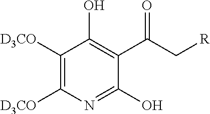

wherein NH is optionally alkylated, sulphur oxidation state will vary S(O) or S(O₂), and R is R′ or R″. In another aspect, the composition is adapted for oral, intravenous, subcutaneous, parenteral, enteral, transcutaneous, transdermal, or rectal administration.

As embodied and broadly described herein, an aspect of the present disclosure relates to a method of making hydrocarbon side chain derivatives of Atpenin A5 comprising:

making hydrocarbon side chain derivatives of Atpenin A5 comprising:

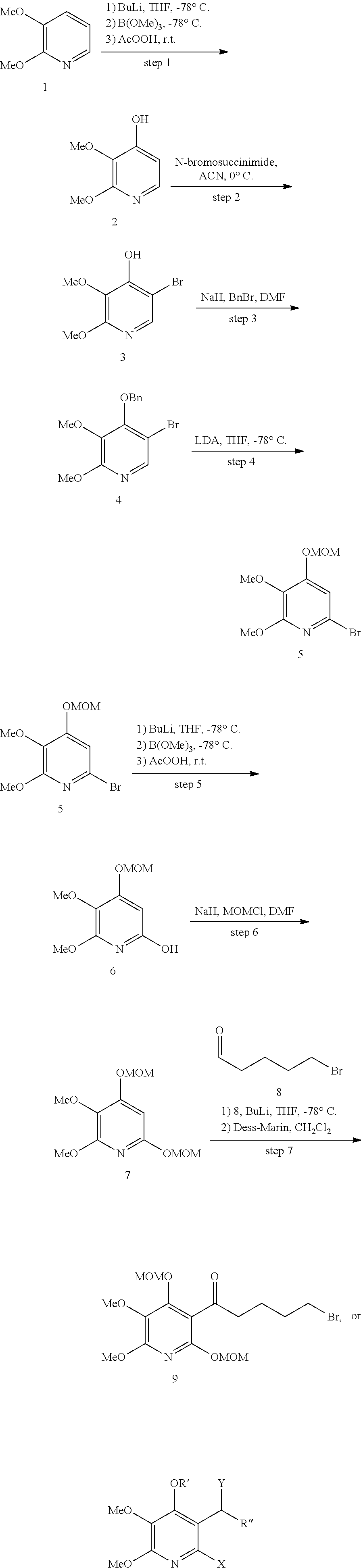

wherein R′ is selected from H, methoxy, methoxymethyl, glycosyl, phosphoryl, alkylamine, polyethylene glycol (PEG), or deuterated; X is selected from OH, methoxy, methoxymethyl, O-methoxymethyl, carbonyl, or alcohol; Y is O; and R″ is selected from H, butyl, pentyl, hexyl, heptyl, octyl, nonyl, decyl, or dodecyl, that are saturated or unsaturated, PEG, or triphenylphosphate; optionally one or both MeO groups are deuterated; and N is N-oxide or N-alkyl. In one aspect, the method further comprises:

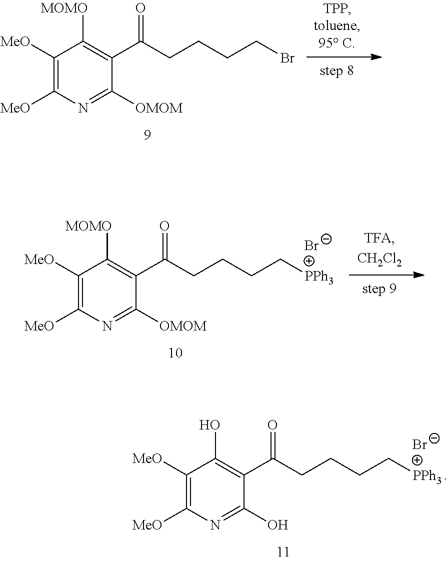

In another embodiment, the present invention includes a method of treating a cancer cell comprising: contacting the cancer cell with an effective amount of a composition comprising an Atpenin 5 derivative selected from:

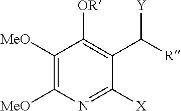

wherein R′ is selected from H, methoxy, or methoxymethyl; X is selected from H, OH, methoxy, or methoxymethyl or O-methoxymethyl; Y is O; and R″ is selected from H, OH, 2-furan, ethyl, propyl, pentyl, hexyl, heptyl, octyl, nonyl, decyl, or dodecyl, that are saturated or unsaturated. In one aspect, the composition comprises at least one of:

| Compound | R′ | X | Y | R″ |
|---|---|---|---|---|
| 16a | H | OH | O | 2-furan |
| 16b | H | OH | O | butyl |
| 16c | H | OH | O | pentyl |
| 16d | H | OH | O | 5-OBn butyl |
| 16e | H | OH | O | 5-hydroxybutyl |
| 16f | H | OH | O | hexyl |
| 16g | H | OH | O | heptyl |
| 16h | H | OH | O | octyl |
| 16i | H | OH | O | nonyl |
| 16j | H | OH | O | decyl |
| 16k | H | OH | O | dodecyl |
| 17 | MOM | OMOM | OH | pentyl |
| 18 | H | OH | OH | pentyl |
| 19 | H | H | OH | pentyl |
| 20 | H | H | O | pentyl |
| 24 | H | H | OMe | Pentyl |

In another aspect, the composition comprises:

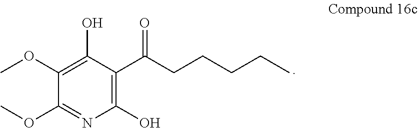 [SMILES: CCCCCC(=O)c1c(O)nc(OC)c(OC)c1O]

In another aspect, the composition comprises:

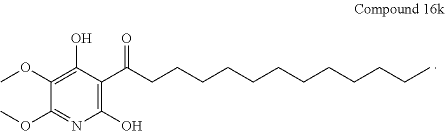 [SMILES: CCCCCCCCCCCCC(=O)c1c(O)nc(OC)c(OC)c1O]

In another aspect, the composition is adapted for oral, intravenous, subcutaneous, parenteral, enteral, transcutaneous, transdermal, or rectal administration. In another aspect, the cancer cell is a prostate cancer cell. In another aspect, the method further comprises adding one or more pharmaceutically acceptable carriers, excipients, buffers, or salts to the composition.

In another embodiment, the present invention includes a composition for treating a cancer cell comprising an effective amount of a composition comprising an Atpenin 5 derivative selected from:

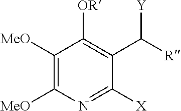

wherein R′ is selected from H, methoxy, or methoxymethyl, wherein X is selected from H, OH, methoxy, or methoxymethyl or O-methoxymethyl, Y is O, and R″ is selected from H, OH, 2-furan, ethyl, propyl, pentyl, hexyl, heptyl, octyl, nonyl, decyl, or dodecyl, that are saturated or unsaturated. In one aspect, the composition comprises at least one of:

| Compound | R′ | X | Y | R″ |
|---|---|---|---|---|
| 16a | H | OH | O | 2-furan |
| 16b | H | OH | O | butyl |
| 16c | H | OH | O | pentyl |
| 16d | H | OH | O | 5-OBn butyl |
| 16e | H | OH | O | 5-hydroxybutyl |
| 16f | H | OH | O | hexyl |
| 16g | H | OH | O | heptyl |
| 16h | H | OH | O | octyl |
| 16i | H | OH | O | nonyl |
| 16j | H | OH | O | decyl |
| 16k | H | OH | O | dodecyl |
| 17 | MOM | OMOM | OH | pentyl |
| 18 | H | OH | OH | pentyl |
| 19 | H | H | OH | pentyl |
| 20 | H | H | O | pentyl |
| 24 | H | H | OMe | Pentyl |

In another aspect, the composition comprises:

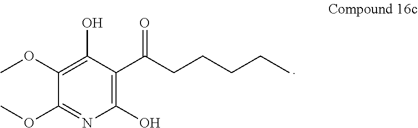 [SMILES: CCCCCC(=O)c1c(O)nc(OC)c(OC)c1O]

In another aspect, the composition comprises:

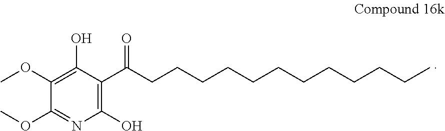 [SMILES: CCCCCCCCCCCCC(=O)c1c(O)nc(OC)c(OC)c1O]

In another aspect, the composition is adapted for oral, intravenous, subcutaneous, parenteral, enteral, transcutaneous, transdermal, or rectal administration. In another aspect, the composition further comprises one or more pharmaceutically acceptable carriers, excipients, buffers, or salts.

In another embodiment, the present invention includes a molecule having the formula:

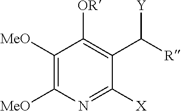

wherein R′ is selected from H, methoxy, or methoxymethyl; X is selected from H, OH, methoxy, or methoxymethyl or O-methoxymethyl; Y is O; and R″ is selected from H, OH, 2-furan, ethyl, propyl, pentyl, hexyl, heptyl, octyl, nonyl, decyl, or dodecyl, that are saturated or unsaturated. In one aspect, the molecule comprises at least one of:

| Compound | R′ | X | Y | R″ |
|---|---|---|---|---|
| 16a | H | OH | O | 2-furan |
| 16b | H | OH | O | butyl |
| 16c | H | OH | O | pentyl |
| 16d | H | OH | O | 5-OBn butyl |
| 16e | H | OH | O | 5-hydroxybutyl |
| 16f | H | OH | O | hexyl |
| 16g | H | OH | O | heptyl |
| 16h | H | OH | O | octyl |
| 16i | H | OH | O | nonyl |
| 16j | H | OH | O | decyl |
| 16k | H | OH | O | dodecyl |
| 17 | MOM | OMOM | OH | pentyl |
| 18 | H | OH | OH | pentyl |
| 19 | H | H | OH | pentyl |
| 20 | H | H | O | pentyl |
| 24 | H | H | OMe | Pentyl |

In another aspect, the molecule has the formula:

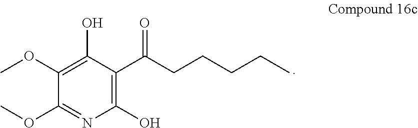 [SMILES: CCCCCC(=O)c1c(O)nc(OC)c(OC)c1O]

In another aspect, the molecule has the formula:

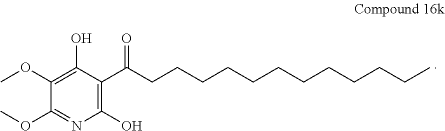 [SMILES: CCCCCCCCCCCCC(=O)c1c(O)nc(OC)c(OC)c1O]

In another aspect, the molecule is adapted for oral, intravenous, subcutaneous, parenteral, enteral, transcutaneous, transdermal, or rectal administration. In another aspect, the molecule is formulated into a composition with one or more pharmaceutically acceptable carriers, excipients, buffers, or salts.

In another embodiment, the present invention includes a method of making hydrocarbon side chain derivatives of atpenin A5 comprising:

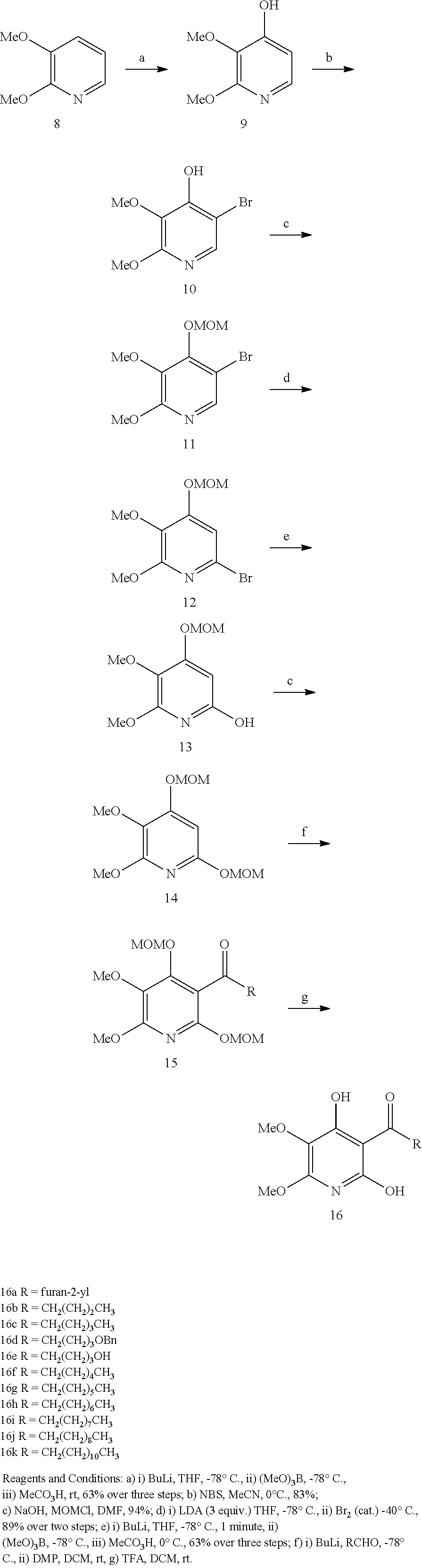

In another embodiment, the present invention includes a method of making oxidation state derivatives of atpenin A5 comprising:

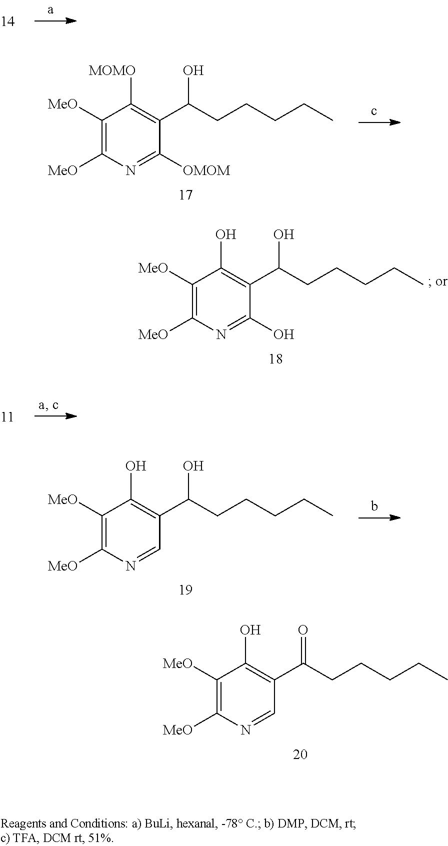

In another embodiment, the present invention includes a method of making a methyl ether derivative of atpenin A5 comprising:

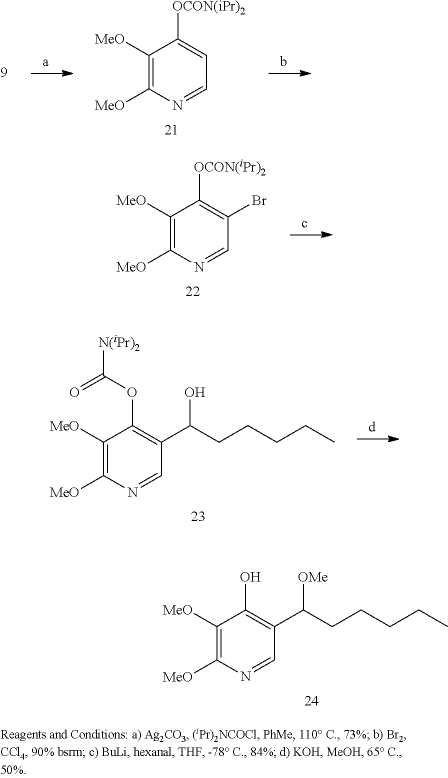

In one embodiment, the present invention includes a compound for use in the treatment of stromal cell proliferation, the compound comprising: an Atpenin 5 derivative selected from:

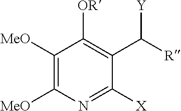

wherein R′ is selected from H, methoxy, or methoxymethyl, wherein X is selected from H, OH, methoxy, or methoxymethyl or O-methoxymethyl, Y is O, and R″ is selected from H, OH, 2-furan, ethyl, propyl, pentyl, hexyl, heptyl, octyl, nonyl, decyl, or dodecyl, that are saturated or unsaturated. In one aspect, the composition comprises at least one of:

| Compound | R′ | X | Y | R″ |
|---|---|---|---|---|
| 16a | H | OH | O | 2-furan |
| 16b | H | OH | O | butyl |
| 16c | H | OH | O | pentyl |
| 16d | H | OH | O | 5-OBn butyl |
| 16e | H | OH | O | 5-hydroxybutyl |
| 16f | H | OH | O | hexyl |
| 16g | H | OH | O | heptyl |
| 16h | H | OH | O | octyl |
| 16i | H | OH | O | nonyl |
| 16j | H | OH | O | decyl |
| 16k | H | OH | O | dodecyl |
| 17 | MOM | OMOM | OH | pentyl |
| 18 | H | OH | OH | pentyl |
| 19 | H | H | OH | pentyl |
| 20 | H | H | O | pentyl |
| 24 | H | H | OMe | Pentyl. |

In another aspect, the composition comprises:

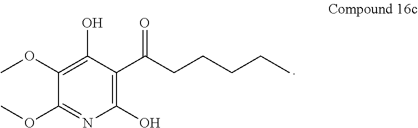 [SMILES: CCCCCC(=O)c1c(O)nc(OC)c(OC)c1O]

In another aspect, the composition comprises:

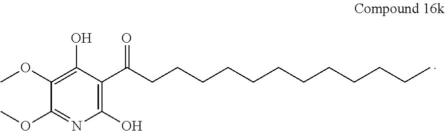 [SMILES: CCCCCCCCCCCCC(=O)c1c(O)nc(OC)c(OC)c1O]

In another aspect, the composition is adapted for oral, intravenous, subcutaneous, parenteral, enteral, transcutaneous, transdermal, or rectal administration. In another aspect, the method or composition further comprises adding one or more pharmaceutically acceptable carriers, excipients, buffers, or salts to the composition. In another aspect, the stromal cells support prostate or kidney cancer cell growth.

In another embodiment, the present invention includes a composition for treating method of treating chronic obstructive pulmonary disease comprising: an effective amount of a composition comprising an Atpenin 5 derivative of Formula:

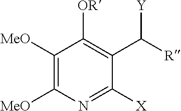

wherein R′ is selected from H, methoxy, methoxymethyl, glycosyl, phosphoryl, alkylamine, polyethylene glycol (PEG), or deuterated; X is selected from OH, methoxy, methoxymethyl, O-methoxymethyl, carbonyl, or alcohol; Y is O; R″ is selected from H, pentyl, hexyl, heptyl, octyl, nonyl, decyl, or dodecyl, that are saturated or unsaturated, PEG or triphenylphosphate group; optionally one or both MeO groups are deuterated; N is N-oxide or N-alkyl; and one or more pharmaceutically acceptable carriers, excipients, buffers, or salts

### BRIEF DESCRIPTION OF THE DRAWINGS

For a more complete understanding of the features and advantages of the present invention, reference is now made to the detailed description of the invention along with the accompanying figures and in which:

FIG. 1 shows the structures of known complex II inhibitors of the prior art.

FIGS. 2A to 2D show modeling of selected AA5 derivatives in the ubiquinone binding site of porcine heart mitochondria complex II (PDB ID: 3AEE). FIG. 2A, hypothetical binding interactions of CII inhibitor 16c. FIG. 2B, overlay of AA5 (7) (turquoise) and 16c (gold) in active site of porcine heart mitochondria complex II. FIG. 2C, hypothetical binding interactions of inactive compound 18. FIG. 2D, hypothetical binding interactions of inactive compound 24 (gold), overlayed with AA5 (7) (turquoise). Red; oxygen, blue; nitrogen, white; hydrogen, gold; carbon. Green dotted lines represent hydrogen bond (greater opacity represents stronger bond).

FIGS. 3A to 3F show complex II inhibitors are potent anti-proliferative agents. FIG. 3A) Dose-response curve of effect of complex II inhibitor 16c on percentage cell viability of DU-145 prostate cancer cells. FIG. 3B, dose-response curve of effect of complex II inhibitor 16c on percentage cell viability of PC3 prostate cancer cells. FIG. 3C, dose-response curve of effect of complex II inhibitor 16c on percentage cell viability of 22Rv1 prostate cancer cells. FIG. 3D, dose-response curve of effect of complex II inhibitor 16c on percentage cell viability of low tumorigenic HEK293 cells. FIG. 3E, dose-response curve of the effect of structurally similar but inactive complex II inhibitor 24 on percentage cell viability of DU-145 prostate cancer cells. FIG. 3F, dose-response curve of the effect of clinical chemotherapeutic enzalutamide on percentage cell viability of 22Rv1 prostate cancer cells after 72 hour incubation. Cell viability measured after 48-hour incubation of compound unless otherwise noted. Values are the mean ±S.D. of triplicate experiments.

FIGS. 4A and 4B show complex II inhibitors with greater potency convey greater cytotoxicity. FIG. 4A, dose-response curve of the effect of complex II inhibitor 16k on percentage cell viability of 22Rv1 prostate cancer cells. FIG. 4B, dose-response curve of effect of complex II inhibitor 16k on the percentage cell viability of low tumorigenic HEK293 cells. Cell viability measured after 48-hour incubation of compound. Values are the mean ±S.D. of triplicate experiments.

FIG. 5 shows the cytotoxic effect of the clinical chemotherapeutic etoposide, complex II inhibitor 16c and inactive control 18 in PC3 prostate cancer cells under hypoxia. Cell death was measured with Annexin V and Sytox Green. Values are the mean ±S.D. for triplicate experiments. A one-way ANOVA analysis was used to compare statistical difference between etoposide and 16c at 1 μM concentrations, p=0.0068.

FIGS. 6A to 6D show complex II inhibitor 16c blocks mitochondrial respiration and function in 22Rv1 prostate cancer cells. FIG. 6A, oxygen consumption rate (OCR), FIG. 6B, basal respiration, FIG. 6C, ATP production, and FIG. 6D, maximal respiration are reduced in a dose-dependent manner. Error bars represent mean ±S.D. n=3.

FIG. 7 shows portions of the inhibitor that are modified to improve the complex II inhibitors of the present invention.

FIGS. 8A to 8C show 3D models of the molecule in the binding pocket, FIG. 8A AA5, FIG. 8B 4-OMe, and FIG. 8C shows the N-oxide in accordance to the molecules in FIG. 7.

FIG. 9 is a graph which shows that the inhibitor 16c potentiates Enzalutamide (ENZA) in LNCaP cells.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

A potent CII inhibitor would be expected to elicit greater anticancer efficacy and would be an excellent candidate for development as a chemotherapeutic agent and as a probe compound for further study of the role of mitochondria in cancer and other diseases. For example, accumulation of the TCA cycle metabolite succinate is an important pathologic event in tissue ischemia, and this is thought to occur via reverse operation of CII.^{[18]} As such, it has been shown that inhibiting CII with low potency compounds, ^{[18-19]} as well as Atpenin A5,^{[20]} is capable of protecting the heart against ischemic reperfusion injury, thereby ascribing CII as a potential target for drug development in stroke. While CII mutations contribute to the pathophysiology of a number of diseases,^{[21]} the pharmacological inhibition of CII by a number of compounds, including clinically approved drugs, has not been linked to increased susceptibility to these diseases.^{[22]}

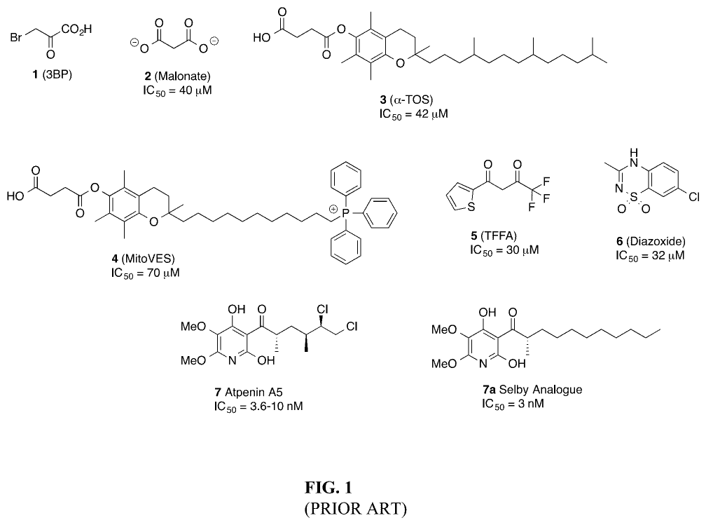

The natural product atpenin A5 (AA5, 7, FIG. 1) is a potent and specific CII inhibitor with an IC₅₀=3.6-10 nM.^{[20, 23]} Inhibition of CII by AA5 results in production of ROS.^{[24]} AA5 and several naturally occurring analogues demonstrate antineoplastic activity in DU-145 prostate cancer cells.^{[25]} However, atpenin B, a close analogue of AA5, was found to have limited anticancer effect in in vivo studies attributed to poor absorption, distribution, metabolism and excretion (ADME) properties.^{[26]} AA5 derivative 7a was prepared by Selby et. al. as part of an antifungal library and gives bovine CII IC₅₀=3 nM.^{[27]} However, the compound retains a stereocenter on the side chain alpha to the ketone. This moiety is present in all of the most active CII inhibitors described in the Selby library. Excision of the methyl resulted in significant amelioration of CII inhibition, suggesting a pharmacophoric role and complicating the generation of synthetic derivatives.

The inhibitory potency for CII and the observed in vitro anticancer effects make AA5 an excellent compound for further drug discovery efforts. However, its suitability as a chemotherapeutic is hindered by low abundance, complex structure, 50-fold reduction in in vivo activity and, until the present invention, incomplete assignment of its pharmacophore.

However, despite these developments, more effective inhibitors of CII/SQR are needed. The present inventors have developed novel Atpenin A5 derivatives. Two of these Atpenin A5 derivatives, termed 16c and 16k, are able to inhibit cancer growth in vitro with a 10-fold increase in efficacy relative the clinically available drug, Enzalutamide.

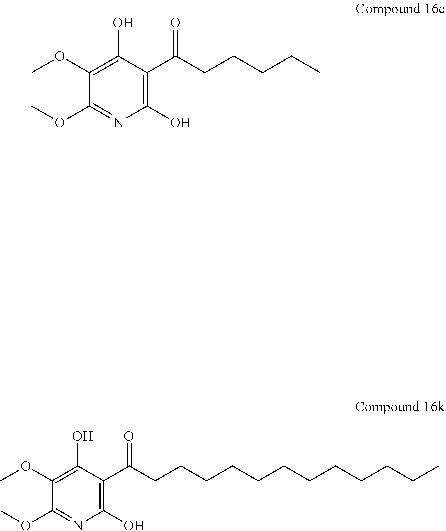

A dosage unit for use of the one or more Atpenin 5 derivative(s) of the present invention, may be a single compound or mixtures thereof with other compounds. The compounds may be mixed together in a manner that forms ionic or even covalent bonds. The Atpenin 5 derivative(s) of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the one or more Atpenin 5 derivative(s) are provided to a patient in need of therapy that includes the need treatment of a cancer. The Atpenin 5 derivative(s) may also be administered as any one of known salt forms.

Atpenin 5 derivative(s) is/are typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the Atpenin 5 derivative(s) is/are may be formulated to provide, e.g., maximum and/or consistent dosing for the particular form for oral, rectal, topical, intravenous injection or parenteral administration. While the Atpenin 5 derivative(s) is/are may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference, and the like, relevant portions incorporated herein by reference.

For example, the Atpenin 5 derivative(s) may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The Atpenin 5 derivative(s) of the present invention may be administered in the form of liposome delivery systems, e.g., small unilamellar vesicles, large unilamellar vesicles, and multilamellar vesicles, whether charged or uncharged. Liposomes may include one or more: phospholipids (e.g., cholesterol), stearylamine and/or phosphatidylcholines, mixtures thereof, and the like.

Atpenin 5 derivative(s) may also be coupled to one or more soluble, biodegradable, bioacceptable polymers as drug carriers or as a prodrug. Such polymers may include: polyvinylpyrrolidone, pyran copolymer, polyhydroxylpropylmethacryl amide-phenol, polyhydroxyethylasparta-midephenol, or polyethyleneoxide-polylysine substituted with palmitoyl residues, mixtures thereof, and the like. Furthermore, the Atpenin 5 derivative(s) may be coupled one or more biodegradable polymers to achieve controlled release of the Atpenin 5 derivative(s), biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the Atpenin 5 derivative(s) and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water-soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagous, trachea, lungs and alveoli, the Atpenin 5 derivative(s) may also be delivered as an intranasal form via use of a suitable intranasal vehicle. For dermal and transdermal delivery, the Atpenin 5 derivative(s) may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of Atpenin 5 derivative(s) may include the following forms.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 mL contain 100 mg of finely divided active ingredient, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment of cancer, which comprise one or more containers containing a pharmaceutical composition comprising a therapeutically effective amount of the Atpenin 5 derivative(s). Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

As used herein, the term “chewable” refers to the Atpenin 5 derivative(s) formulated into semi-soft, palatable and stable chewable treats for use without the addition of water. It should be appreciated to the skilled artisan that a chewable composition will be stable and palatable, fast disintegrating, semi-soft medicated chewable tablets (treats) by extrusion without the addition of extraneous water. A soft chewable tablet does not harden on storage and are resistant to microbial contamination. A semi-soft chewable contain a blend of any one or more of binders, flavors, palatability enhancers, humectants, disintegrating agents, non-aqueous solvents, and diluents that are plasticized with liquid plasticizers, such as glycols and polyols to make them ductile and extrudable. The chewable can be made by extrusion, e.g., including fats or lipids as plasticizers and binding agents, is manufactured in the absence of added water, uses plasticizers to replace water in extrudable matrices, contains humectants to maintain the extrudable chew in a pliant and soft state during its shelf life, or any combination thereof. The chewable form may be provided in conjunction with one or more flavorants and/or taste masking agents that improve the taste of the formulation greater than 10, 20, 30, 40, 50, 60, 70, 80, or 90%. The chewable can include the active agent and the ion exchange resin to enhance taste masking.

In certain embodiments, the Atpenin 5 derivative(s) of the present invention can be formulated into a dosage form in which the final formulation includes no other active agent. In such an embodiment, the Atpenin 5 derivative(s) is/are provided such that only non-active excipients, carriers, etc., that are pharmacologically acceptable and without any other active agent, which shall be used with the phrase “consisting essentially of” requires the specified integer(s) or steps as well as those that do not materially affect the character or function of the claimed invention. In other embodiments, the Atpenin 5 derivative(s) may be the only active agent provided in a simple carrier, in which case the composition is said to “consist” of the Atpenin 5 derivative(s) without any other agents, and when used in a method, the Atpenin 5 derivative(s) will be said to be included in a formulation “consisting” of the active Atpenin 5 derivative(s).

The inventors have shown that these derivatives are able to inhibit the proliferation of stromal cells within the microenvironment of prostate cancers. Their data suggests that compounds 16c and 16k are able to prevent not only prostate tumor cell (22Rv1) growth but prevent the growth of the underlying stromal cells (WPMY-1) as well. This disruption of the tumor microenvironment using 16c or 16k may be the basis for the development for a new anticancer treatment. It was found that that 16c and 16k are able to combat prostate cancer by inhibiting proliferation in the underlying stromal cells that support the tumors.

The benefits of the present invention includes, e.g., they inhibit the growth of stromal cells that support tumors, there is a greater efficacy when compared to current methods of treatment (Enzalutamide), it serves to circumvent anticancer drug resistance, and is selective for cancer cells and their associated stroma.

Chemistry. The known crystal structure of E. coli CII with AA5,^{[28]} and porcine CII (PDB ID: 3AEE) indicates a role for the highly substituted pyridine ring in active site recognition and hence in the first series of designed analogues this moiety remained unchanged. The hydrocarbon side chain of AA5 contains three stereocenters that hinder large-scale preparation, similarly AA5 derivative 7a (FIG. 1), while simplified, still contained a stereocenter alpha to the side chain ketone. The inventors sought to simplify the side chain through the generation of unfunctionalized and non-chiral hydrocarbon chain derivatives.

Several hydrocarbon chain derivatives of AA5 have been synthesized to evaluate antifungal activity.^{[27]} However, only one has shown enhanced activity. Three total synthesis of the AA5 scaffold have been reported in the literature from the groups of Omura,^{[29]} Selby,^{[27]} and Carreira^{[30]} as well as one report of racemic atpenin B by Quéguiner et. al.^{[31]} The synthetic strategy to access AA5 derivatives for structure-activity relationship (SAR) study is based on elements from all four routes.

Commercially available 2,3-dimethoxypyridine (8) was lithiated with ⁿbutyl lithium and exposed to trimethylborate, followed by oxidation with peracetic acid to provide 2,3 -dimethyoxy-4-hydroxypyridine (9) in moderate yield (Scheme 1). Regioselective addition of bromine was achieved at the 5-position by exposure to N-bromosuccinimide and purification by column chromatography to afford 5-bromo-2,3-dimethoxypyridin-4-ol (10) as the major product. Subsequent protection as the methoxymethyl ether (11) was achieved in excellent yield. Rearrangement of the bromine of pyridine 11 from the 5-position to the 6-position was accomplished by a ‘halogen dance’ reaction upon exposure to LDA and catalytic bromine, providing 6-bromo-pyridine (12) in excellent yield. Metal-halogen exchange with ⁿbutyl lithium, critically with just one minute of stirring at −78° C., followed by trapping with trimethyl borate^{[32]} and subsequent oxidation with peracetic acid provided 5,6-dimethoxy-4-(methoxymethoxy)pyridin-2-ol (13). Protection of the hydroxy functionality of 13 as the methoxymethyl ether afforded the critical intermediate 2,3-dimethoxy-4,6-bis(methoxymethoxy)pyridine (14) in excellent yield. Subsequent exposure of pyridine (14) to ⁿbutyl lithium and a suitably functionalized aldehyde provided the respective alcohol which was oxidized to the ketone with DMP to afford a series of methoxymethyl ether protected atpenin A5 derivatives of type 15. Deprotection by exposure to TFA resulted in a variety of hydrocarbon chain atpenin A5 derivatives (16a-k) in moderate to good yield.

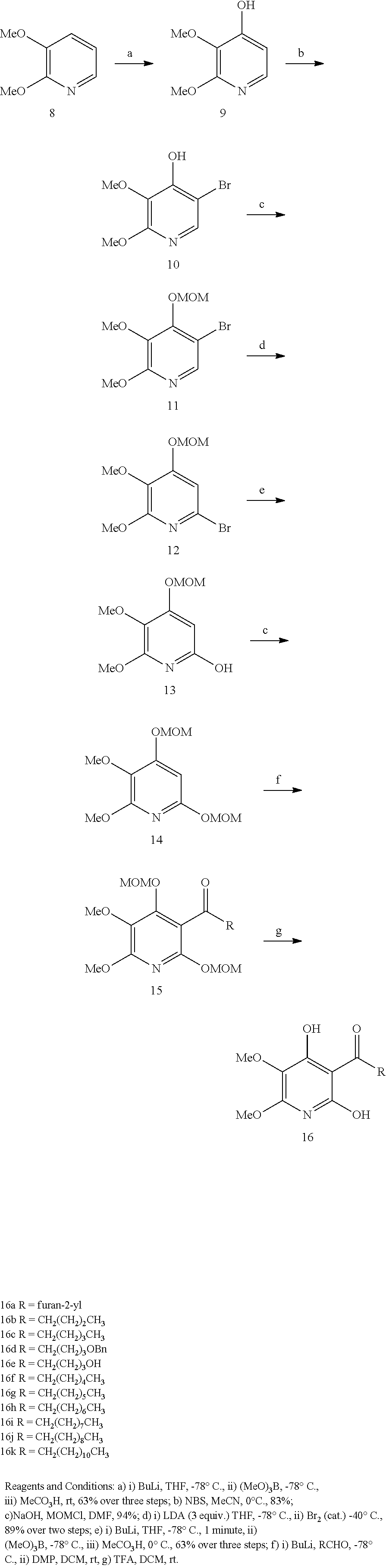

Scheme 1. Synthesis of hydrocarbon side chain derivatives of atpenin A5.

To determine the effect of the oxidation state of the first position of the hydrocarbon chain derivatives on CII inhibition, triol (18) (Scheme 2) was isolated from the methoxymethyl ether intermediate (17) by omitting the oxidation step from the developed synthetic route.

Given the structural similarity between AA5 and the natural CII substrate ubiquinone, the inventors hypothesized that the 2,3-dimethoxy and 4-hydroxy substituents on the AA5 pyridine ring would be essential for CII binding site recognition. However, as the 6-hydroxy substituent of AA5 is not present in ubiquinone the inventors sought to determine the effects of removing this group. To this end, the inventors synthesized the 6-deshydroxy derivative (20) by direct addition of a suitably functionalized aldehyde to bromide (11), lacking the 6-hydroxy functionality (Scheme 2). This route provided diol intermediate (19) in good yield which was selectively oxidized with DMP to provide the desired 1-(4-hydroxy-5,6-dimethoxypyridin-3-yl)hexan-1-one (20) in moderate yield.

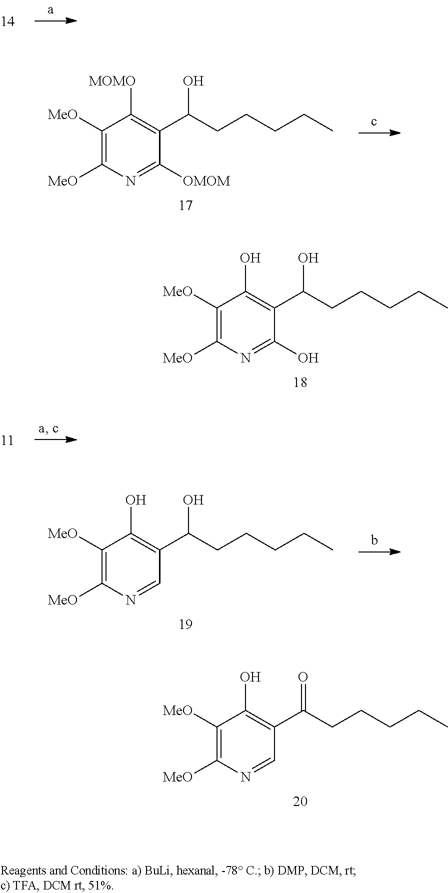

Scheme 2. Synthesis of oxidation state derivatives of atpenin A5.

During the course of this investigation into the synthesis of AA5 derivatives the inventors partially adopted the route of Quéguiner et. al. to atpenin B.^{[31]} Protection of alcohol (9) as the N,N-diisopropyl carbamate (21) followed by addition of Br₂ provided access to bromide (22) in excellent yield (Scheme 3). Subsequent transmetallation and trapping with hexanal provided alcohol (23) in good overall yield. However, deprotection of the N,N-diisopropyl carbamate by exposure to methanolic potassium hydroxide failed to yield the desired compound, with the respective methyl ether derivative (24) obtained in 50% yield.

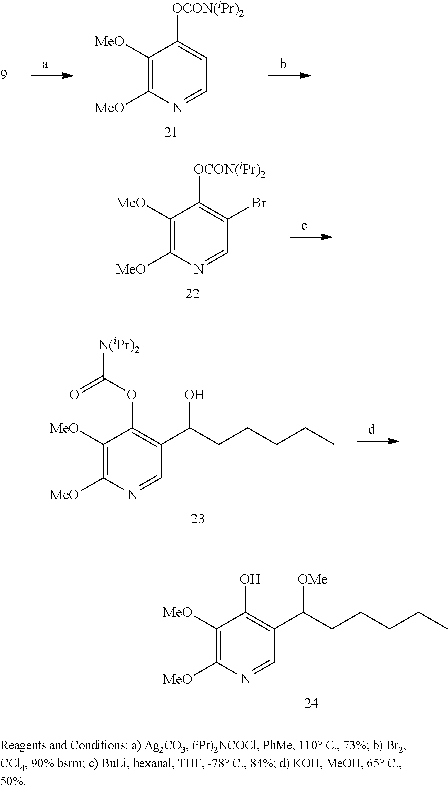

Scheme 3. Synthesis of a methyl ether derivative of Atpenin A5.

Complex II inhibition assay. Complex II activity was measured spectrophotometrically according to a literature method using isolated rat heart mitochondria, with suitable modifications to ensure rapid isolation.^{[11]} The control compound AA5 was determined to have an IC₅₀=3.3 nM, in accordance with the 3.6-10 nM reported in the literature,^{[20, 23]} the clinically approved agent Diazoxide has a literature IC₅₀=32 μM and is included in Table 1 for comparative purposes of CII inhibitors of non-AA5 structure.^{[16]} With the sole exception of the furan-2-yl moiety, all of the synthesized side chain derivatives displayed rat heart CII inhibition activity ranging from 3.3 nM to 3.7 μM. Compounds 16j and 16k display CII inhibition activity of IC₅₀ 8.6 and 3.3 nM respectively, two of the most potent inhibitors ever described. To compare CII species differences directly, two compounds were synthesized that were included in a recently described antifungal library (16b & 16f),^{[27]} which demonstrate bovine CII IC₅₀=96 nM and 20 nM, respectively. In rat heart CII, activity is substantially attenuated with IC₅₀=346 nM and 282 nM, respectively. Therefore, suggesting that compound 16k described herein, may represent the most potent CII inhibitor yet described.

TABLE 1
Complex II inhibition activity, ClogP, Mw, PSA and Lipophilic efficiency of
AA5 derivatives of the present invention.

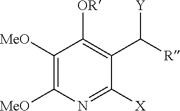

| | | | | | | | | | CII IC₅₀ |
|---|---|---|---|---|---|---|---|---|---|
| Compound | R′ | X | Y | R″ | Mw | ClogP^{a} | PSA^{b} | LLE^{c} | (nm)^{d} |
| 16a | H | OH | O | 2-furan | 265.22 | −0.02 | 102.02 | n/a | 0%^{e} |
| 16b | H | OH | O | butyl | 255.27 | 1.09 | 88.88 | 5.37 | 345.5 |
| 16c | H | OH | O | pentyl | 269.29 | 1.53 | 88.88 | 5.62 | 64 |
| 16d | H | OH | O | 5-OBn butyl | 361.39 | 2.02 | 98.11 | 3.41 | 280.8 |
| 16e | H | OH | O | 5- | 271.27 | −0.35 | 109.11 | 6.90 | 3730.2 |
| | | | | hydroxybutyl | | | | | |
| 16f | H | OH | O | hexyl | 283.32 | 1.98 | 88.88 | 4.57 | 282.4 |
| 16g | H | OH | O | heptyl | 297.35 | 2.42 | 88.88 | 4.89 | 49.1 |
| 16h | H | OH | O | octyl | 311.37 | 2.87 | 88.88 | 4.93 | 15.7 |
| 16i | H | OH | O | nonyl | 325.40 | 3.31 | 88.88 | 4.69 | 9.9 |
| 16j | H | OH | O | decyl | 339.43 | 3.75 | 88.88 | 4.32 | 8.6 |
| 16k | H | OH | O | dodecyl | 367.48 | 4.64 | 88.88 | 3.76 | 3.3 |
| 17 | MOM | OMOM | OH | pentyl | 359.42 | 2.95 | 88.5 | n/a | 0% |
| 18 | H | OH | OH | pentyl | 271.31 | 0.97 | 92.04 | n/a | 0% |
| 19 | H | H | OH | pentyl | 255.31 | 2.24 | 71.81 | n/a | 0% |
| 20 | H | H | O | pentyl | 253.29 | 2.97 | 68.65 | n/a | 0% |
| 24 | H | H | OMe | pentyl | 269.34 | 2.88 | 60.81 | n/a | 0% |
| Atpenin A5 | n/a | n/a | n/a | n/a | 366.24 | 2.64 | 88.88 | 5.36 | 3.3 |
| Diazoxide | n/a | n/a | n/a | n/a | 230.67 | 1.0 | 58.53 | 1.85 | 32,000^{[16]} |

^{a}Calculated by MarvinSketch 5.10.3
^{b}Polar Surface Area (pH = 7.4), calculated by MarvinSketch 5.10.3
^{c}Ligand-Lipophilicity Efficiency (LLE = pIC₅₀-ClogP)
^{d}Mean value of four experiments
^{e}0% inhibition of CII at 100 nM concentration

Addition of an aromatic furnan-2-yl moiety (16a) to the 5-position of the AA5 pyridine ring abrogates CII inhibition activity. Potentially due to the presence of the extended conjugated system. Shortening the side chain to a simple butyl chain provided a compound with an IC₅₀ of 345 nM and a 1.5-fold lower ClogP (16b). Interestingly, this derivative when assayed in bovine CII provided an IC₅₀=96 nM.^{[27]} Retaining the length of the natural AA5 hydrocarbon chain but with no substituents, resulted in compound 16c which displays an IC₅₀ of 64 nM, lower lipophilicity, but identical PSA to 16b. The ligand-lipophilicity efficiency (LLE) of 16c, a measure of potency and lipophilicity, was calculated to be 5.62, the optimum value obtained across all of the derivatives herein. A LLE greater than 5, combined with a lipophilic ClogP is often considered desirable for a lead or clinical compound.^{[33]}

Retaining the length of the natural product side chain but replacing the terminal methyl with a benzyl ether (16d) reduced activity (IC₅₀=281 nM) compared with AA5. Removal of the benzyl protecting group to provide alcohol (16e) further decreased activity providing a CII IC₅₀ of 3.7 μM, but still provided a compound of 10-fold greater activity than diazoxide. This observation suggests the alcohol moiety of 16e is not engaging in hydrogen bonding within the complex II protein, given its lower potency compared with the identical side chain length derivative 16c. The importance of lipophilicity in the design of CII inhibitors is also supported by this data.

Addition of a methyl group to the pentyl side chain of 16c provided hexyl derivative 16f which displayed equipotent activity with the benzyl ether derivative (16d) with an IC₅₀ of 282 nM, which by way of explanation, and in no way a limitation of the present invention, further suggests that the oxygen of 16d and 16e is not involved in intermolecular interactions with CII. Again, a difference in activity between species of CII is observed, when derivative 16f is assayed in bovine CII an IC₅₀=20 nM is obtained.^{[27]} This shows that the CII inhibitors detailed herein may possess much greater activity when assayed in bovine CII.

Homologation from hexyl through to dodecyl resulted in a progressive increase in CII inhibition resulting in the identification of three compounds with highly potent CII inhibition activity; 16i which displays IC₅₀=9.9 nM, 16j with IC₅₀=8.6 nM and 16k with IC₅₀=3.3 nM. Homologation was ceased at the dodecyl chain (16k) as this compound represents comparable molecular weight and PSA to the AA5 natural product, albeit with increased ClogP. In general, increasing lipophilicity increases potency, a common phenomenon in medicinal chemistry. Compound 16f however, does not fit this trend when going from pentyl (16c) to heptyl (16g) wherein the activity of hexyl side chain derivate 16f displays attenuated CII inhibition activity.

A similar trend is observed in the MitoVES (4, FIG. 1) series of compounds wherein shortening the alkyl chain results in progressive loss of CII inhibition activity.^{[14]} Penetration of the mitochondrial inner membrane to access the CII target is necessary for inhibitory activity, with lipophilicity a key determinant in this process along with cationic charge.^{[34]} To achieve greater delivery to the mitochondria the inventors can attach a mitochondrial targeting triphenylphosphonium (TPP) group. However, the optimum hydrocarbon linker length between the active CII inhibitor ‘warhead’ and the TPP group can be obtained.^{[14]}

Computational analysis of the crystal structure of the CII ubiquinone binding site with bound AA5 predicts a pharmacophoric role for the highly substituted pyridine moiety but no role for the 5-position side chain carbonyl in intermolecular binding.^{[28]} Next, the inventors generated analogues of the AA5 derivatives about the ketone moiety to confirm this model. The intermediate hexanol derivative 17 displays 0% inhibition of CII at 100 nM concentration, suggesting that both pyridine alcohols are required for intermolecular binding to the CII quinone site, or alternatively, the additional steric bulk of the MOM groups precludes access of the compound to the CII binding site. Interestingly, alcohol 18, an oxidation state analogue of the active CII inhibitor 16c displayed no CII inhibition activity. Excision of the pyridine 6-position alcohol (19) also resulted in an inactive compound. Oxidation of alcohol 19 to the respective carbonyl (20) and thus a direct bioisostere of 16c wherein the pyridine 6-positon alcohol is excised, displayed no activity at 100 nM concentration. Methyl ether 24, like its alcohol counterpart, showed no activity to inhibit CII at 100 nM. Together these observations suggest a pharmacophoric role for the carbonyl of the side chain in the design of AA5 derivatives. While the possibility that the inactivity of these derivatives may indicate a failure of the compounds to penetrate into the mitochondria inner membrane, analysis of their physicochemical properties disputes this. PSA values of 18 and 24 (71.81 and 60.81) calculated at pH 7.4 are significantly lower than active inhibitor 16c and the AA5 natural product (both 88.88). ClogP values, a direct measure of lipophilicity, of 18 and 24 (2.24 and 2.88) are higher than the active and structurally similar inhibitor 16c (1.53) and in the case of compound 24, higher than the AA5 scaffold (2.64) itself, although shape, size and charge requirements also play a role in mitochondrial penetration.

Molecular modeling. Through detailed SAR studies the inventors have shown that the side chain ketone of AA5 is critical for CII inhibition activity and thus constitutes part of the pharmacophore of this class of inhibitor. Quantitative structure-activity relationship prediction was employed to understand the SAR of the compound series. The known crystal structure of AA5 bound in the ubiquinone binding site of porcine heart mitochondria complex II (PDB ID: 3AEE) provided the basis of the analysis. The AA5 binding site was defined and the ligand replaced with compounds 16c, 18 or 24 using SeeSAR 5.5 (BioSolveIT GmbH). Binding poses for each ligand were generated and scoring performed by HYDE.^{[35]}

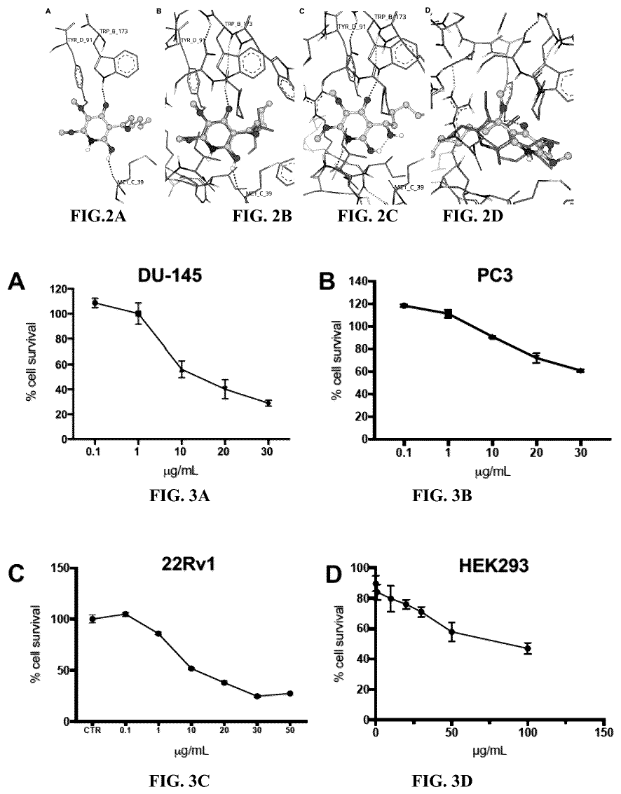

Active CII inhibitor 16c is predicted to form hydrogen bond interactions between the pyridine 4-OH of 16c to the amine of the TrpB173 amino acid residue, between the 3 -OMe and the hydroxyl moiety of TyrD91 and between the pyridine 6-OH and the hydroxy moiety of MetC39 (FIG. 2A). This is in agreement with the observed inactivity of the 6-deshydroxy or protected analogues 17, 19, 20 and 24. These predicted active site interactions match those obtained from the crystal structure of AA5 with porcine heart mitochondria CII (PDB ID: 3AAE), when 16c is overlaid with AA5 (FIG. 2B).

Quantitative structure-activity relationship (QSAR) prediction of the inactive alcohol derivative 18 shows a significant change of binding pose, which is sufficient to prevent formation of, or reduce the strength of, hydrogen bond interactions between the pyridine 3-OMe and TyrD91 and between the pyridine 6-OH and MetC39. The 6-OH hydrogen is predicted instead to partake in intramolecular hydrogen bonding to the side chain alcohol, forming a thermodynamically stable six-membered ring (FIG. 2C). The methyl ether derivative 24, lacking the pyridine 6-OH, undergoes a significant change in binding pose compared with AA5 (FIG. 2D) resulting in only hydrogen bond interactions involving the pyridine 4-OH moiety and thus loss of CII inhibition activity.

The QSAR ligand-protein interaction predictions show excellent correlation with the determined SAR. The molecular modelling studies and SAR data reveal a pharmacophoric role for the side chain ketone moiety, not as might be expected by participating in hydrogen bonding to CII binding site residues, but by conferring a bioactive conformation to active inhibitors. Indeed, in all three compound interaction models (16c, 18 and 24, FIGS. 2A to 2D) and in the original crystal structure of AA5 bound with porcine heart mitochondria CII, the ketone oxygen is not predicted to be involved in intermolecular hydrogen bonding. Excision of the carbonyl moiety leads to conformational changes that reduce distances between hydrogen bond donors and receptors and leads to suboptimal bonding angles.

FIGS. 2A to 2D show modeling of selected AA5 derivatives in the ubiquinone binding site of porcine heart mitochondria complex II (PDB ID: 3AEE). FIG. 2A, hypothetical binding interactions of CII inhibitor 16c. FIG. 2B, overlay of AA5 (7) (turquoise) and 16c (gold) in active site of porcine heart mitochondria complex II. FIG. 2C, hypothetical binding interactions of inactive compound 18. FIG. 2D, hypothetical binding interactions of inactive compound 24 (gold), overlayed with AA5 (7) (turquoise). Red; oxygen, blue; nitrogen, white; hydrogen, gold; carbon. Green dotted lines represent hydrogen bond (greater opacity represents stronger bond).

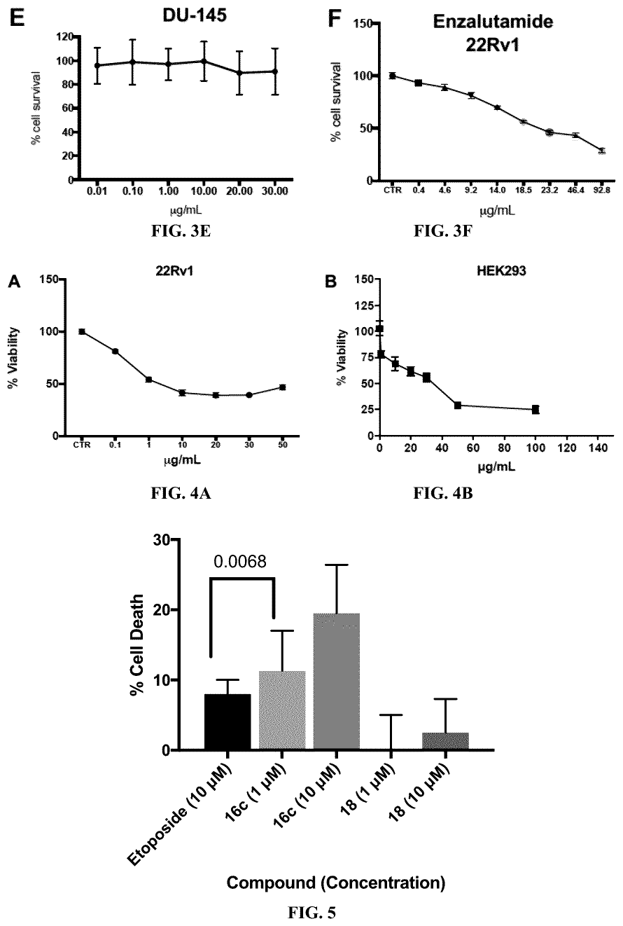

Cytotoxicity assay. Derivative 16c was taken forward for cytotoxicity evaluation. This derivative was chosen for further study due to its structural resemblance to natural AA5, same PSA, yet lower lipophilicity engendering greater solubility, structural simplicity and favorable LLE value (5.62) combined with CII inhibition potency (IC₅₀=64 nM, 17.23×10⁻³ μg/mL). Three human prostate cancer cell lines and HEK293 cells were exposed to compound 16c and cytotoxicity measured by MTS assay after 48 hours (FIGS. 3A-F).

Treatment of DU-145 prostate cancer cells with CII inhibitor 16c provided a significant inhibitory effect on cell growth in a dose-dependent manner, resulting in an IC₅₀ of 12 μg/mL (FIG. 3A). When inhibitor 16c was exposed to PC3 prostate cancer cells a lower inhibitory effect was observed, resulting in an IC₅₀ of 38 μg/mL (FIG. 3B). In 22Rv1 prostate cancer cells, inhibitor 16c demonstrated an IC₅₀ of 11 μg/mL (FIG. 3C). Selectivity to cancerous cells over low tumorigenic cells, was confirmed by the use of human embryonic kidney cells (HEK293) where 16c demonstrated an IC₅₀ of 81 μg/mL, a 7.4-fold selectivity for DU-145 and 22Rv1 prostate cancer cells over this transformed cell line (FIG. 3D). The inactive control compound 24 displayed no growth inhibition effect upon treatment of DU-145 cells up to 30 μg/mL (FIG. 3E), implicating CII inhibition as a primary mechanism of action to inhibit prostate cancer cell growth. The clinically available prostate cancer drug enzalutamide displayed an IC₅₀ of 21 μg/mL after 72 hour treatment in 22Rv1 prostate cancer cells (FIG. 3F), a cell line known to be resistant to this chemotherapeutic. CII inhibitor 16c displayed an IC₅₀ of 11 μg/mL after just 48 hours. This observed cytotoxicity, along with that of CII inhibitor 16k (FIGS. 4A-B), suggests that CII inhibition may be a novel target for the treatment of advanced drug resistance prostate cancer.

Further, when the more potent CII inhibitor 16k (IC50=3.3 nM, 1.21×10⁻³ μg/mL) was exposed to 22Rv1 prostate cancer cells, an even greater reduction of cell viability was observed with an IC₅₀ of just 2 μg/mL (FIG. 4A). This shows that CII inhibition potency is directly proportional to anti-proliferative effect. However, maximal cytotoxicity is reached at 10 μg/mL, beyond which a plateau is observed. Selectivity over the low tumorigenic HEK293 cell line was again observed with 16k providing an IC₅₀ of 35 μg/mL, conferring 17.5-fold selectivity (FIG. 4B). A summary of the IC₅₀ values for compounds 16c and 16k across prostate cancer cell lines, prostate stroma, HEK293 cells and 3T3 cells compared with enzalutamide positive control is depicted in table 2.

TABLE 2
Activity of CII inhibitors 16c and 16k, the inactive control 24 and
the clinical chemotherapeutic enzalutamide across a panel of human
prostate cancer cells, prostate stromal cells, low tumorigenic human
endothelial kidney cells and mouse embryonic fibroblast cells.

| | DU-145 | PC3 | 22Rv1 | WPMY-1 | HEK293 | 3T3 |
|---|---|---|---|---|---|---|
| | IC₅₀ | IC₅₀ | IC₅₀ | IC₅₀ | IC₅₀ | IC₅₀ |
| Compound | (μg/mL) | (μg/mL) | (μg/mL) | (μg/mL) | (μg/mL) | (μg/mL) |
| 16c | 12 | 38 | 11 | 18.4 | 81 | 31.8 |
| 16k | N.D.^{a} | N.D. | 2 | 10.7 | 35 | N.D. |
| 24 | >30^{b} | N.D. | ND | N.D. | N.D. | N.D. |
| Enzalutamide | N.D. | N.D. | 21 | 96.9 | N.D. | 70.8 |

^{a}Not Determined
^{b}No cell-death at 30 μg/mL concentration.

To determine if the synthetic CII inhibitors retain the selectivity of the parent compound for CII over other constituent members of the electron transport chain the inventors assayed 16c and 16K for mitochondrial complex I (CI) inhibition activity. Both compounds were found to be inactive up to 10 μM leading to a selectivity ratio for CII:CI of >156-fold for 16c and >3030-fold for 16k (Table 3). This data highlights these compounds as promising chemical probes for further interrogation of the NADH-fumarate reductase system, specifically targeting complex II.

TABLE 3
Selectivity of compounds 16c, 16k and AA5 for inhibition
of mitochondrial complex II over complex I.

| | Complex II | Complex I | Selectivity Ratio |
|---|---|---|---|
| Compound | IC₅₀ (nM) | IC₅₀ (nM) | (CII:CI) |
| 16c | 64 | >10,000 | >156 |
| 16k | 3.3 | >10,000 | >3030 |
| 7 (AA5) | 3.3 | >10,000 | >3030 |

A recent report identified AA5 as an active hit in a screen for the identification of compounds that target cells in dormant tumor spheroid regions.^{[37]} Cancer cells in poorly vascularized tumor regions react to diminished nutrients by stopping cell cycle progression and becoming dormant. Hence, those cells in hypoxic regions of the tumor are more likely to be resistant to chemotherapeutics. Utilizing a microfluidic culture system,^{[38]} the inventors exposed PC3 prostate cancer cells under hypoxic conditions to the clinical chemotherapeutic etoposide, CII inhibitor 16c and inactive control 18 (FIG. 5). As expected, the inactive compound 18 demonstrated no cytotoxic effect up to 10 μM concentration within experimental error, further supporting the antineoplastic role of the CII inhibition mechanism. Complex II inhibitor 16c provided 12% cell death at 1 μM concentration and 20% cell death at 10 μM concentration. Etoposide is known to have poor cytotoxicity in hypoxic environments,^{[39]} displaying only 7% cell death at 10 μM concentration in hypoxic assay. The designed CII inhibitor (16c), at 10-fold lower concentration, provides superior cytotoxicity in PC3 cells under hypoxia than etoposide.

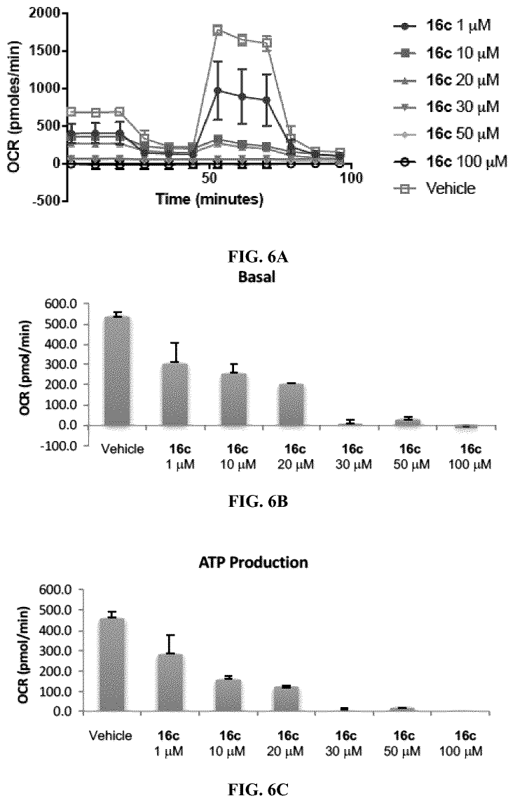

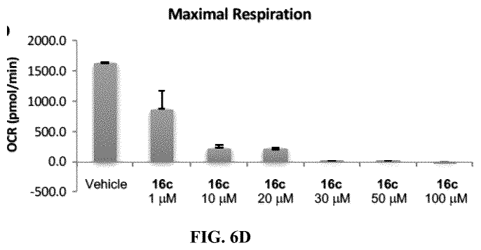

Next, the inventors determined the effect of complex II inhibition by the developed inhibitors on mitochondrial metabolic processes. The effect of 16c on electron transport parameters of 22Rv1 cells after 48 hour incubation was studied using a Seahorse XF Extracellular Flux Analyzer. The oxygen consumption rate of 22Rv1 cells is substantially reduced in a dose-dependent manner upon exposure to 16c (FIG. 6A). CII inhibitor 16c significantly inhibited mitochondrial function in 22Rv1 prostate cancer cells in a dose-dependent manner, including reducing basal respiration (FIG. 6B), inhibition of ATP production (FIG. 6C) and reduction of maximal respiration (FIG. 6D) compared with no treatment control.

Thus, the present inventors disclose the design, synthesis and evaluation of highly potent CII inhibitors. Analysis of physicochemical properties indicated compound 16c as the most ‘drug-like’ molecule for further study, while the potency of 16c is reduced (CII IC₅₀=64 nM) over 16k (CII IC₅₀=3.3 nM) the ligand-lipophilicity efficiency of 16c is the most optimal of the CII inhibitors synthesized and represents far greater potency than existing inhibitors while retaining ‘drug-like’ properties and selectivity for mitochondrial complex II over mitochondrial complex I. Significant anti-proliferative activity was demonstrated by 16c in DU-145 and 22Rv1 human prostate cancer cell lines. This effect was shown to be selective with a 7.4-fold greater anti-proliferative activity over low tumorigenic human embryonic kidney cells. Both compounds 16c and 16k showed superior anti-proliferative activity to the clinically approved chemotherapeutic enzalutamide. Further, this effect is retained under hypoxic conditions. Inhibitor 16c significantly inhibited mitochondrial electron transport parameters, reducing oxygen consumption rate, basal respiration, ATP production and maximal respiration.

In summary, for the first time, truly potent CII inhibitors with nanomolar activity are shown to elicit significant and selective anti-proliferative activity. CII inhibitors 16c, 16j and 16k represent valuable molecular tools to study the effect of CII in cancer and other diseases.

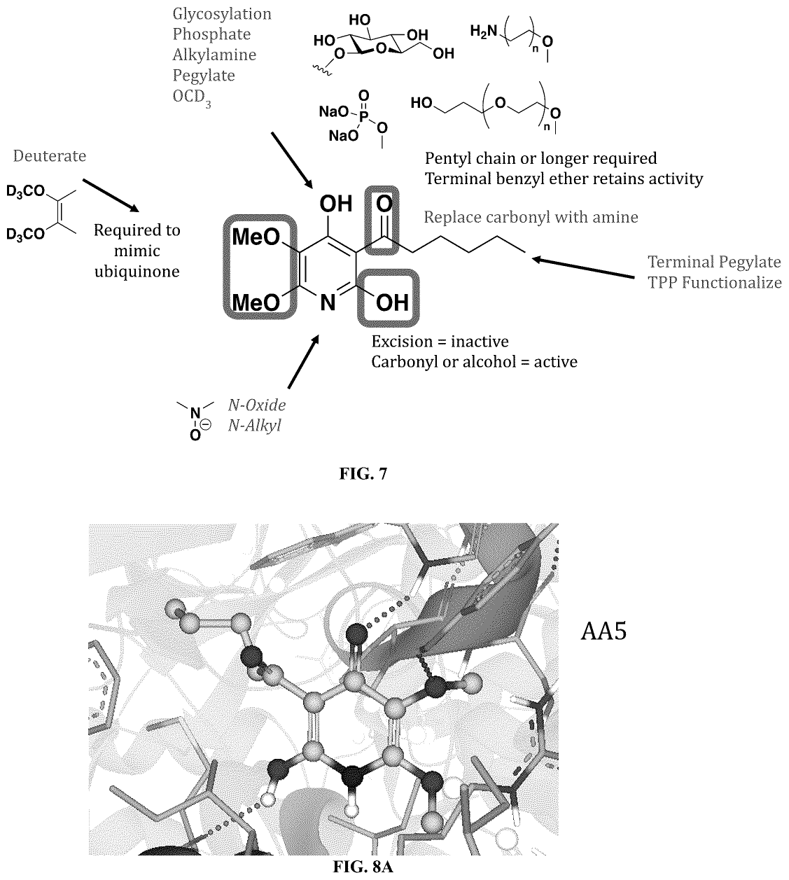

FIG. 7 shows portions of the inhibitor that can be modified to improve the complex II inhibitors of the present invention. The molecule can be modified by deuterating the portions that mimic ubiquinone. The N can be converted to an N-oxide or N-alkyl. The carbonyl can be replaced with an amino, and/or a pentyl or longer chain is attached that can have a terminal benzyl ether to retain activity, and/or it can be terminally PEGylated or TPP functionalized. Finally, the hydroxy group can be glycosylated, phosphorylated, alkylaminated, PEGylated or deuterated. The other hydroxyl group can be converted to a carbonyl or alcohol but not excised.

TABLE 4
shows the PK profile (in vivo) of the following molecule at 1 mg/Kg:

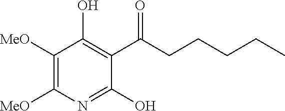 [SMILES: CCCCCC(=O)c1c(O)nc(OC)c(OC)c1O]

mCII IC₅₀ = 64 nM
mCI IC₅₀ =>10,000 nM

| | | λ_{z} | t_{1/2λz} | tₘₐₓ | Cₘₐₓ | AUC_{inf,obs} | CL_{obs} | AUC_{inf,pred} | CL_{pred} | Vss_{obs} | Vss_{pred} |
|---|---|---|---|---|---|---|---|---|---|---|---|
| Gender | Rsq | (h⁻¹) | (h) | (h) | (ng/ml) | (ng · h/mL) | (ml/h/kg) | (ng · h/mL) | (ml/h/kg) | (ml/kg) | (ml/kg) |
| M | 0.88 | 0.41 | 1.71 | 0.50 | 23.94 | 10.68 | 93637.60 | 10.60 | 94352.64 | 134647.72 | 129169.94 |
| F | 0.98 | 0.40 | 1.73 | 0.25 | 140.13 | 35.95 | 27812.86 | 35.98 | 27793.15 | 15089.80 | 15275.19 |
| F* | 0.98 | 0.40 | 1.73 | 0.25 | 76.70 | 23.61 | 42363,46 | 23.63 | 42317.75 | 29000.69 | 29417.48 |
| F** | 0.98 | 0.40 | 1.73 | 0.50 | 13.31 | 8.30 | 120529.64 | 8.32 | 120160.32 | 179378.36 | 182145.61 |

M = male; F = female; Rsq = goodness of fit statistic for the terminal (log-linear) phase between the linear regression and the data; λ_{z} = first order rate constant associated with the terminal (log-linear) portion of the curve; t_{1/2λz} = terminal half-life; Tₘₐₓ = time of maximum observed concentration; Cₘₐₓ = maximum observed concentration; AUC_{inf} = area under the concentration curve from dosing time extrapolated to infinity; CL = clearance; Vss = volume of distribution at steady state; obs = based on observed data; pred = based on predicted λ_{z}.
* One data point 267 ng/ml at t = 0.25 h was excluded (included points are 83.3 and 70.1 ng/ml).
** All three data points at t = 0.25 h were excluded.

tₘₐₓ: 0.38 hrs, Cₘₐₓ: 56.02 ng/mL, AUC: 19.64 ng.h/mL, t_{1/2}: 102.6 mins, and Vd: 89,529.14 mL/Kg.

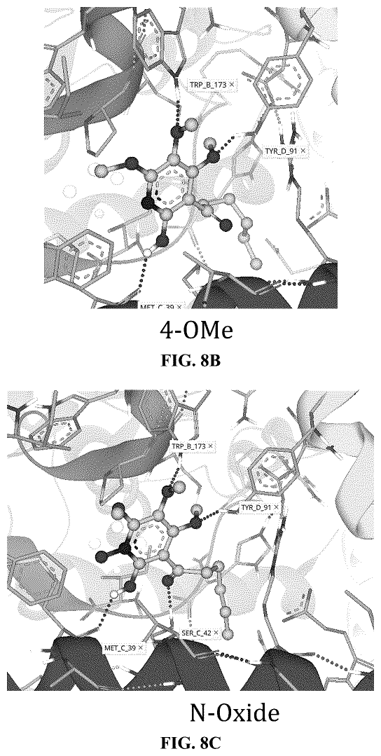

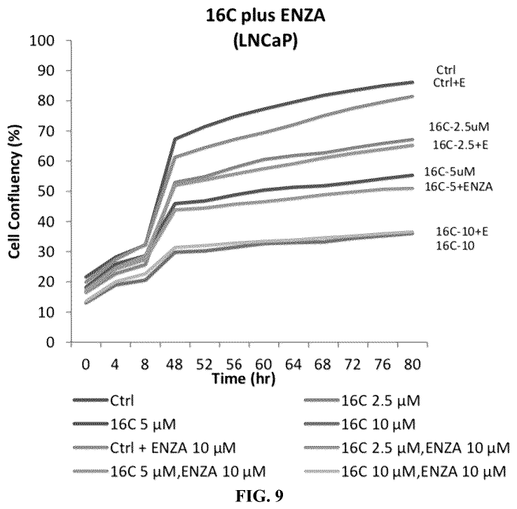

FIGS. 8A to 8C show 3D models of the molecule in the binding pocket, FIG. 8A AA5, FIG. 8B 4-OMe, and FIG. 8C shows the N-oxide in accordance to the molecules in FIG. 7.

FIG. 9 is a graph which shows that the inhibitor 16c potentiates Enzalutamide (ENZA) in LNCaP cells.

The following molecules were also synthesized.

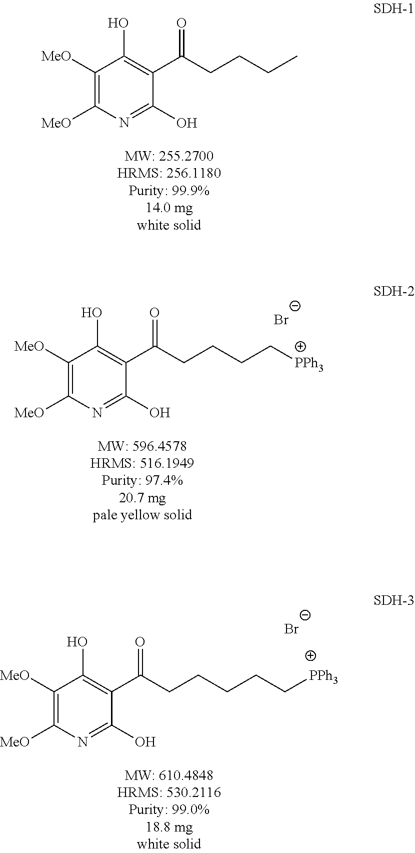

TABLE 5

| Compound | Control | 1 uM | 100 nM |
|---|---|---|---|
| SDH1 | 100% | 35.13% | 65.17% |
| SDH2 | 100% | 43.53% | 61.64% |

The results with SDH1 show that linker length can be changed to shorter and longer as well as using polyethylene glycol. The results with SHD2 show that triphenylphosphate (TPP) functionalization retains activity to increase localization to the mitochondria.

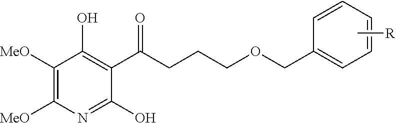

Shows that the linker may be increased or decreased, R=2, 3 or 4 mono, di, tri substitution with halogen electron-donating group or electron-withdrawing groups, and/or the ether Oxygen may be NH, S, or C.

Finally, the following compounds can also fit in the pocket as shown.

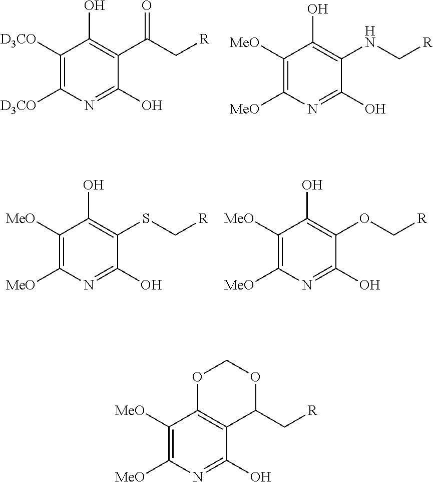

wherein R is any side group, NH may be alkylated, and sulphur oxidation state will vary S(O) or S(O₂).

Chemistry. All reactions were carried out in oven- or flame-dried glassware under positive nitrogen pressure unless otherwise noted. Reaction progress was monitored by thin-layer chromatography (TLC) carried out on silica gel plates (2.5 cm×7.5 cm, 200 μm thick, 60 F254) and visualized by using UV (254 nm) or by potassium permanganate and/or phosphomolybdic acid solution as indictor. Flash column chromatography was performed with silica gel (40-63 μm, 60 Å) or on a Teledyne Isco (CombiFlash R_{f} 200 UV/Vis). Commercial grade solvents and reagents were purchased from Fisher Scientific (Houston, TX) or Sigma Aldrich (Milwaukee, WI) and were used without further purification except as indicated. Anhydrous solvents were purchased from Across Organics and stored under and atmosphere of dry nitrogen over molecular sieves.

¹H, ¹³C, COSY, HMQC and DEPT NMR spectra were recorded in the indicated solvent on a Bruker 400 MHz Avance III HD spectrometer at 400 and 100 MHz for ¹H and ¹³C respectively with TMS as an internal standard. Multiplicities are indicated by s (single), d (doublet), dd (doublet of doublets), t (triplet), q (quartet), m (multiplet), br (broad). Chemical shifts (δ) are reported in parts per million (ppm), and coupling constants (J), in hertz. High-resolution mass spectrometry was performed on a LC/MS IT-TOF (Shimadzu) using an ESI source conducted at the University of Texas at Arlington, Shimadzu Center for Advanced Analytical Chemistry. High-pressure liquid chromatography was performed on a Gilson HPLC system with 321 pumps and 155 UV/Vis detector using trilution software v2.1 with an ACE Equivalence 3 (C18, 3 μM, 4.6×150 mm) column. All samples were determined to possess >95% purity.

2,3-dimethoxypyridin-4-ol (9).^{[29}] To a solution of 2,3-dimethoxypyridine (0.30 g, 2.16 mmol) in THF (15 mL) at −78° C. was added ⁿBuLi (1.3 mL, of a 2.5 M solution in hexanes, 3.24 mmol). The mixture was stirred for 1 h at 0° C. in an ice-water bath. The reaction mixture was cooled to −78° C., B(OMe)₃ (0.62 mL, 5.4 mmol) was added and the mixture stirred at −78° C. for 2 h. A solution of peracetic acid (32 wt % in dilute acetic acid, 1.02 mL, 4.32 mmol) was added and the mixture was slowly warmed to room temperature over one hour. To the reaction solution was added an aqueous solution of sodium bisulfite. The solution was extracted with ethyl acetate, washed with brine and dried over sodium sulfate, the solvent was removed in vacuo and purification by flash column chromatography (DCM:Et₂O 94:6) afforded the title compound as a colorless oil (211 mg, 63%): ¹H NMR (400 MHz, CDCl₃) δ3.71 (s, 3H), 3.90 (s, 3H), 6.48(d, 1H, J=5.7 Hz), 7.61 (d, 1H, J=5.7 Hz), 7.99 (s, 1H); ¹³CNMR (100 MHz, CDCl₃) δ53.30, 60.20, 107.30, 130.40, 140.70, 156.60, 157.80.

5-bromo-2,3-dimethoxypyridin-4-ol (10). To a solution of 2,3-dimethoxypryidin-4-ol (0.106 g, 0.68 mmol) in MeCN (5 mL) at 0° C. was added NBS (0.134 g, 0.75 mmmol). The reaction mixture was allowed to warm to room temperature and stirred overnight. The reaction mixture was extracted with ethyl acetate, the organic layer washed with brine and dried over sodium sulfate, the solvent was removed in vacuo and purification by flash column chromatography (Hexane:EtOAc 20:1) afforded the title compound as a yellow powder (0.132 g, 83%): ¹H NMR (400 MHz, CDCL₃) δ3.91 (s, 3H), 3.99 (s, 3H), 6.77 (s, 1H),7.90 (s, 1H); ¹³C NMR (100 MHz, CDCl₃) δ53.76, 60.99, 100.88, 130.63, 141.98, 152.60, 156.59; HRMS (ESI) m/z calcd for C₇H₈BrNO₃ (M+Na⁺): 255.9580, found 255.9574.

5-bromo-2,3-dimethoxy-4-(methoxymethoxy)pyridine (11). To a solution of 5-bromo-2,3-dimethoxypyridin-4-ol (10) (3.68 g, 15.7 mmol) in DMF (50 mL) at 0° C. was added NaH (0.943 g, 23.6 mmol of a 60% dispersion in mineral oils). The mixture was stirred for 10 minutes and methoxymethyl chloride (1.79 ml, 23.6 mmol) was added. The reaction mixture was allowed to warm to room temperature and stirred for 1 h. The reaction mixture was extracted with ethyl acetate, the organic layer washed with brine and dried over sodium sulfate, the solvent was removed in vacuo and purification by flash column chromatography (Hexane:EtOAc 20:1) afforded the title compound as a yellow powder (4.11 g, 94%): ¹H NMR (400 MHz, CDCl₃) δ3.57(s, 3H), 3.82 (s, 3H), 3.94 (s, 3H), 5.33 (s, 2H), 7.90 (s, 1H); ¹³C NMR (100 MHz, CDCl₃) δ53.88, 57.68, 60.51, 98.57, 107.24, 136.29, 142.25, 153.16, 158.56; HRMS (ESI) m/z calcd for C₉H₁₂NBrO₄ (M+Na⁺) 299.9842 found 299.9835.

6-bromo-2,3-dimethoxy-4-(methoxymethoxy)pyridine (12). Lithium diisopropylamide (LDA) was prepared by addition of ⁿBuLi (1.41 mL, 1.6 M in hexanes, 2.26 mmol) to diisopropylamine (0.317 mL, 2.26 mmol) in THF (10 mL) at −78° C. and warmed up to 0° C. for 30 min with stirring. LDA solution was cooled to −78° C. and a solution of 5-bromo-2,3-dimethoxy-4-(methoxymethoxy)pyridine (11) (0.21 g, 0.76 mmol) in THF (10 mL) was added. The solution was warmed to −40° C. in an acetonitrile-dry ice bath and 2 uL of bromine was added. The reaction mixture was stirred for 1 h at −40° C. and then cooled to −78° C. An excess of EtOH (4 mL) was added and the mixture was warmed up to 23° C., which was treated with a saturated aqueous solution of NH₄Cl (5 mL) and then extracted with ethyl acetate. The organic washings were washed with brine and dried over sodium sulfate, the solvent was removed in vacuo and purification by flash column chromatography (Hexane:EtOAc 10:1) afforded the title compound as a yellow powder (0.187 g with 89%): ¹H NMR (400 MHz, CDCl₃) δ3.39 (s, 3H), 3.72 (s, 3H), 3.86 (s, 3H), 5.14 (s, 2H), 6.82 (s, 1H); ¹³C NMR (100 MHz, CDCl₃) δ54.06, 56.30, 60.36, 94.42, 108.95, 131.10, 131.69, 158.5, 157.43; HRMS (ESI) m/z calcd for C₉H₁₂NBrO₄ (M+Na⁺) 299.9842 found 299.9839.

5,6-dimethoxy-4-(methoxymethoxy)pyridin-2-ol (13). To a solution of 6-bromo-2,3-dimethoxy-4-(methoxymethoxy)pyridine (12) (3.79 g, 13.6 mmol) in THF (120 mL) at −78° C. was quickly added BuLi (18.7 mL, of a 1.6 M solution in hexanes, 40.9 mmol). The mixture was stirred for 1 minute and B(MeO)₃ (4.89 mL, 40.9 mmol) was added with stirring for 2 h at −78° C. A solution of peracetic acid (32 wt % in dilute acetic acid, 12.9 mL, 54.5 mmol) was then added and the reaction mixture allowed to warm to 0° C. under stirring for 2 h. An aqueous solution of sodium hydrogenosulfite was added dropwise, and the mixture extracted with ethyl acetate. The organic washings were washed with brine and dried over sodium sulfate, the solvent was removed in vacuo and purification by flash column chromatography (Hexane:EtOAc 10:1) afforded the title compound as a yellow powder (1.86 g, 63%): ¹H NMR (400 MHz, CDCl₃) δ3.41(s, 3H), 3.69 (s, 3H), 3.86 (s, 3H), 5.16 (s, 2H), 6.04 (s, 1H); ¹³C NMR (100 MHz, CDCl₃) δ54.90, 56.45, 60.99, 89.02, 94.36, 126.31, 155.69, 158.34, 160.32; HRMS (ESI) m/z calcd for C₉H₁₃NO₅ (M+Na⁺) 238.0686 found 238.0678

2,3-dimethoxy-4,6-bis(methoxymethoxy)pyridine (14). To a solution of 5,6-dimethoxy-4-(methoxymethoxy)pyridin-2-ol (13) (1.805 g, 8.38 mmol) in DMF (10 mL) at 0° C. was added NaH (0.537 g , 13.4 mmol, of a 60% dispersion in mineral oils). After 10 minutes, methoxymethyl chloride (0.96 mL, 12.6 mmol) was added. The reaction mixture was allowed to warm to room temperature and stirred for 1 h. The reaction mixture was extracted with ethyl acetate, the organic washings were washed with brine and dried over sodium sulfate, the solvent was removed in vacuo and purification by flash column chromatography (Hexane:EtOAc 10:1) afforded the title compound as a yellow powder (2.05 g, 94%): ¹H NMR (400 MHz, CDCl₃) δ3.48 (s, 3H), 3.49 (s, 3H), 3.77 (s, 3H), 3.92 (s, 3H), 5.22 (s, 2H), 5.41 (s, 2H), 6.21 (s, 1H); ¹³C NMR (100 MHz, CDCl₃) δ53.67, 56.47, 56.96, 60.86, 88.99, 91.95, 94.54, 127.50, 156.02, 156.46, 159.21; HRMS (ESI) m/z calcd for C₁₁H₁₇NO₆ (M+Na⁺) 282.0948 found 282.0940.

(2,4-dihydroxy-5,6-dimethoxypyridin-3-yl)(furan-2-yl)methanone (16a). General method for the synthesis of compounds 16a-16k. To a solution of 2,3-dimethoxy-4,6-bis(methoxymethoxy)pyridine (14) (0.0290 g, 0.11 mmol) in THF (5 mL) at −78° C. was added ⁿBuLi (0.09 mL, 2.5 M, 0.22 mmol) and the reaction mixture stirred for 1 h. Furan-2-carbaldedhye (10.6 mg, 0.11 mmol) was added and the mixture stirred for 1 h. The reaction was quenched by addition of EtOH and extracted with EtOAC, the organic washings were washed with brine and dried over Na₂SO₄,the solvent was removed in vacuo and the crude product used for the next step without further purification. To a solution of the crude material in CH₂Cl₂ (5 mL) at room temperature was added Dess-Martin Periodinane (0.071 g, 0.17 mmol), and the reaction mixture stirred for 30 minutes. The reaction was quenched by addition of saturated sodium thiosulfate and extracted with EtOAc, the organic washings were washed with brine and dried over Na₂SO₄,the solvent was removed in vacuo and the crude product used for the next step without further purification. To a solution of the crude material in CH₂Cl₂ (2 mL) at 0° C. was added TFA (0.5 mL) and the reaction mixture stirred for 30 minutes. The solvent was removed in vacuo and the residue purified by flash column chromatography (DCM:MeOH 97:3) to afford the title compound as a colorless oil (0.014 g, 48%): ¹H NMR (400 MHz, CDCl₃) δ3.84 (s, 3H), 4.11 (s, 3H), 6.57 (1H, s), 7.68 (2H, m, 2x CH); ¹³C NMR (100 MHz, CDCl₃) δ57.31, 61.54, 111.97, 120.97, 122.16, 146.87, 151.46, 156.19, 160.53, 182.98.

1-(2,4-dihydroxy-5,6-dimethoxypyridin-3-yl)pentan-1-one (16b). A colorless oil (0.015 g, 53%): ¹H NMR (400 MHz, CDCl₃) δ0.96 (3H, t, J=7.0 Hz), 1.33 (m, 2H), 1.66 (m, 2H), 3.05 (2H, t, J=7.0 Hz), 3.79 (s, 3H), 4.16 (s, 3H); ¹³C NMR (100 MHz, CDCl₃) δ13.94, 22.51, 26.32, 42.25, 61.51, 101.04, 121.05, 155.56, 162.07, 205.93; HRMS (ESI) m/z calcd for C₁₂H₁₇NO₅ (M+Na⁺) 278.0999 found 278.1005.

1-(2,4-dihydroxy-5,6-dimethoxypyridin-3-yl)hexan-1-one (16c). A colorless oil (0.037 g, 51%): ¹H NMR (400 MHz, CDCl₃) δ0.89 (3H, t, J=7.0 Hz), 1.30 (m, 4H), 1.65 (m, 2H), 3.04 (2H, t, J=7.0 Hz), 3.80 (s, 3H), 4.16 (s, 3H); ¹³C NMR (100 MHz, CDCl₃) δ14.0, 22.4, 24.07, 31.82, 42.50, 57.78, 60.61, 101.55, 121.06, 155.08, 161.82, 206.60; HRMS (ESI) m/z calcd for C₁₃H₁₉NO₅ (M+Na⁺) 292.1161 found 292.1155.

5-(benzyloxy)-1-(2,4-dihydroxy-5,6-dimethoxypyridin-3-yl)pentan-1-one (16d). A colorless oil (0.098 g, 58%): ¹H NMR (400 MHz, CDCl₃) δ1.77 (m, 4H), 1.25 (m, 18 H), 3.09 (2H, t, J=6.3 Hz), 3.51 (2H, t, J=6.3 Hz), 3.78 (s, 3H), 4.15 (s, 3H), 4.50 (s, 2H), 7.32 (m, 5 H); ¹³C NMR (100 MHz, CDCl₃) δ20.94, 29.37, 42.26, 57.94, 61.49, 70.16, 72.92, 100.67, 121.49, 127.49, 127.60, 128.34, 138.55, 155.94, 161.82, 205.74; HRMS (ESI) m/z calcd for C₁₉H₂₃NO₆ (M+Na⁺) 384.1423 found 384.1417.

1-(2,4-dihydroxy-5,6-dimethoxypyridin-3-yl)-5-hydroxypentan-1-one (16e). A colorless oil (0.045 g, 56%): ¹H NMR (400 MHz, CD3OD) δ1.60 (m, 2H), 1.73 (m, 2 H), 3.10 (2H, t, J =7.4 Hz), 3.59 (2H, t, J=7.4 Hz), 3.71 (s, 3H), 4.99 (s, 3H); ¹³C NMR (100 MHz, CD3OD) δ21.86, 33.26, 43.84, 61.19, 62.75, 101.06, 124.70, 160.08, 162.86, 207.51; HRMS (ESI) m/z calcd for C₁₂H₁₇NO₆ (M+Na⁺) 294.0948 found 294.0954.

1-(2,4-dihydroxy-5,6-dimethoxypyridin-3-yl)heptan-1-one (16f). A colorless oil (0.025 g, 52%): ¹H NMR (400 MHz, CDCl₃) δ0.89 (3H, t, J=7 Hz), 1.31 (m, 6H), 1.66 (m, 2H), 3.03 (2H, t, J=7 Hz), 3.79 (s, 3H), 4.16 (s, 3H); ¹³C NMR (100 MHz, CDCl₃) δ13.94, 22.51, 26.32, 42.25, 57.63, 61.51, 101.04, 121.05, 155.56, 162.07, 205.93; HRMS (ESI) m/z calcd for C₁₄H₂₁NO₅ (M+Na⁺) 306.1312 found 306.1308.

1-(2,4-dihydroxy-5,6-dimethoxypyridin-3-yl)octan-1-one (16g). A colorless oil (0.017 g, 51%): ¹H NMR (400 MHz, CDCl₃) δ0.88 (3H, t, J=7.0 Hz), 1.31 (m, 8H), 1.64 (m, 2H), 3.06 (2H, t, J=7.0 Hz), 3.79 (s, 3H), 4.17 (s, 3H); ¹³C NMR (100 MHz, CDCl₃) δ14.08, 22.64, 24.21, 29.17, 29.39, 31.75, 42.56, 57.63, 61.50, 100.99, 121.25, 155.95, 161.82, 206.33; HRMS (ESI) m/z calcd for C₁₅H₂₃NO₅ (M+Na⁺) 320.1468 found 320.1463.

1-(2,4-dihydroxy-5,6-dimethoxypyridin-3-yl)nonan-1-one (16h). A colorless oil (0.019 g, 55%): ¹H NMR (400 MHz, CDCl₃) δ0.89 (3H, t, J=7.0 Hz), 1.29 (m, 10 H), 1.69 (m, 2H), 3.10 (2H, t, J=7.0 Hz), 3.82 (s, 3H), 4.21 (s, 3H); ¹³C NMR (100 MHz, CDCl₃) δ14.10, 22.67, 24.15, 29.19, 29.34, 29.44, 31.85, 42.77, 58.33, 61.68, 100.48, 121.96, 154.84, 161.67, 206.41; HRMS (ESI) calcd for C₆H25NO₅ (M+Na⁺) 334.1625 found 334.1626.

1-(2,4-dihydroxy-5,6-dimethoxypyridin-3-yl)decan-1-one (16i). A colorless oil (0.018 g, 52%): ¹H NMR (400 MHz, CDCl₃) δ0.87 (3H, t, J=7.0 Hz), 1.26 (m, 12 H), 1.65 (m, 2H), 3.06 (2H, t, J.0=7 Hz), 3.78 (s, 3H), 4.17 (s, 3H); ¹³C NMR (100 MHz, CDCl₃) δ14.10, 22.65, 24.19, 29.29, 29.42, 29.51, 31.87, 42.56, 57.63, 61.52, 100.05, 121.10, 155.19, 161.65, 206.76; HRMS (ESI) m/z calcd for C₇H27NO₅ (M+Na⁺) 348.1781 found 348.1778.

1-(2,4-dihydroxy-5,6-dimethoxypyridin-3-yl)undecan-1-one (16j). A colorless oil (0.028 g, 55%): ¹H NMR (400 MHz, CDCl₃) δ0.87 (3H, t, J=7.0 Hz), 1.26 (m, 14 H), 1.65 (m, 2H), 3.04 (2H, t, J=7.0 Hz), 3.78 (s, 3H), 4.17 (s, 3H); ¹³C NMR (100 MHz, CDCl₃) δ14.09, 22.66, 24.20, 29.32, 29.43, 29.51, 29.55, 29.59, 31.88, 42.57, 57.25, 61.50, 100.69, 121.26, 155.67, 160.99, 206.08; HRMS (ESI) m/z calcd for C₁₈H₂₉NO₅ (M+Na⁺) 362.1938 found 362.1937.

1-(2,4-dihydroxy-5,6-dimethoxypyridin-3-yl)tridecan-1-one (16k). A colorless oil (0.035 g, 56%): ¹H NMR (400 MHz, CDCl₃) δ0.89 (3H, t, J=7.0 Hz), 1.25 (m, 18 H), 1.68 (m, 2H), 3.07 (2H, t, J=7.0 Hz), 3.79 (s, 3H), 4.15 (s, 3H); ¹³C NMR (100 MHz, CDCl₃) δ14.10, 22.67, 24.20, 29.34, 29.42, 29.51, 29.55, 29.64, 29.67, 31.90, 42.55, 57.6361.51, 101.07, 121.25, 155.19, 161.80, 206.42; HRMS (ESI) m/z calcd for C₁₈H₂₉NO₅ (M+Na⁺) 390.2251 found 390.2256.

1-(5,6-dimethoxy-2,4-bis(methoxymethoxy)pyridin-3-yl)hexan-1-ol (17). To a solution of 2,3-dimethoxy-4,6-bis(methoxymethoxy)pyridine (14) (0.030 g, 0.116 mmol) in THF (3 mL) at −78° C. was added nBuLi (0.19 mL, of a 2.5 M solution in hexanes, 0.35 mmol), which was stirred for 1 h. Hexanal (0.074 mL, 0.58 mmol) was added and the reaction stirred for 1 h. The reaction was quenched with ethanol and extracted with ethyl acetate, the organic washings were washed with brine and dried over sodium sulfate, the solvent was removed in vacuo and the residue purified by column chromatography (Hexanes:EtOAc 4:1) to afford the title compound as a colorless oil (33.3 mg, 80%): ¹H NMR (400 MHz, CDCl₃) δ0.89 (t, 3H, J=7.0 Hz), 1.30 (m, 4H), 1.52 (m, 2H), 1.74 (m, 1H),1.91 (m, 1H), 3.22 (s, 1H), 3.53 (s, 3H), 3.56 (s, 3H), 3.75 (s, 3H), 3.93 (s, 3H), 4.95 (t, 1H, J=6.7 Hz), 5.29 (d, 1H, J=5.0 Hz), 5.32 (d, 1H, J=5.0 Hz), 5.49 (d, 1H, J=5.0 Hz), 5.60 (d, 1H, J=5.0 Hz); ¹³C NMR (100 MHz, CDCl₃) δ13.9, 22.2, 25.9, 31.8, 37.5, 53.6, 57.3, 57.8, 60.6, 67.3, 91.8, 99.2, 112.0, 129.0, 153.0, 154.9, 156.8.

(rac)-3-(1-hydroxyhexyl)-5,6-dimethoxypyridine-2,4-diol (18). To a solution of 2,3-dimethoxy-4,6-bis(methoxymethoxy)pyridine (14) (0.020 g, 0.077 mmol) in THF (5 mL) at −78° C. was added ⁿBuLi (0.068 mL, of a 2.5 M solution in hexanes, 0.17 mmol), which was stirred for 1 h. Hexanal (0.010 mL, 0.077 mmol) was added and the reaction stirred for 1 h. The reaction was quenched with ethanol and extracted with ethyl acetate, the organic washings were washed with brine and dried over sodium sulfate, the solvent was removed in vacuo and the crude product used for the next step without further purification. To a solution of the crude material in CH₂Cl₂ (4 mL) at 0° C. was added TFA (1 mL) and the reaction stirred for 30 minutes. The solvent was removed in vacuo and the residue purified by flash column chromatography (DCM:MeOH 97:3) to afford the title compound as a racemic mixture as a colorless oil (0.061 g, 63%): ¹H NMR (400 MHz, CDCl₃) δ0.87 (m, 4H), 1.29 (m, 6H), 3.81 (m, 4H), 3.93 (s, 3H), 5.55 (d, 1H, J=7.0 Hz), 7.71 (br. s, 1H); ¹³C NMR (100 MHz, CDCl₃) δ13.89, 22.63, 25.17, 31.79, 36.94, 72.83, 80.03, 157.16, 163.58; HRMS (ESI) m/z calcd for C₁₃H₂₁NO₅ (M+H−H₂O⁺) 254.1387 found 254.1366.

(rac)-5-(1-hydroxyhexyl)-2,3-dimethoxypyridin-4-ol (19). To a solution of 5-bromo-2,3-dimethoxy-4-(methoxymethoxy)pyridine (11) (0.032 g, 0.115 mmol) in THF (5 mL) at −78° C. was added ⁿBuLi (0.10 mL, of a 2.5 M solution in hexanes, 0.25 mmol), which was stirred for one minute. Hexanal (0.012 g, 0.15 mmol) was added and stirring continued for 1 h. The reaction was quenched by ethanol and extracted with ethyl acetate, the organic washings were washed with brine and dried over sodium sulfate, the solvent was removed in vacuo and the residue purified by flash column chromatography (Hexane:EtOAc 4:1) to afford the title compound as a colorless oil (25 mg, 85%): ¹H NMR (400 MHz, CDCl₃) δ0.89 (m, 2H), 1.30 (m, 6H), 1.73 (q, J=5.5 Hz), 3.90 (s, 3H), 4.27 (s, 3H), 4.86 (br. t, 1H, J=5.5 Hz), 7.76 (s, 1H), 10.59 (br. s, 2H); ¹³C NMR (100 MHz, CDCl₃) δ13.90, 22.45, 25.0, 31.37, 37.0, 58.58, 61.40, 69.54, 114.39, 117.28, 124.97, 130.53, 132.05, 155.08, 162.0.

1-(4-hydroxy-5,6-dimethoxypyridin-3-yl)hexan-1-one (20). To a solution of 5-(1-hydroxyhexyl)-2,3-dimethoxypyridin-4-ol (19) (28 mg, 0.11 mmol) in CH₂Cl₂ (5 mL) at room temperature was added Dess-Martin Periodinane (0.073 g, 0.17 mmol) and the reaction stirred for 30 minutes. The reaction was quenched by addition of saturated sodium thiosulfate and extracted with ethyl acetate, the organic washings were washed with brine and dried over sodium sulfate, the solvent was removed in vacuo and the crude product used for the next step without further purification. To a solution of the crude material in CH₂Cl₂ (4 mL) at 0° C. was added TFA (1 mL) and the reaction stirred for 30 minutes. The solvent was removed in vacuo and the residue purified by flash column chromatography (DCM:MeOH 97:3) to afford the title compound as a colorless oil (0.015 g, 51%): ¹H NMR (400 MHz, CDCl₃) δ0.91 (3H, t, J=7.0 Hz), 1.37 (m, 6H), 1.75 (m, 2H), 2.93 (2H, t, J=7.0 Hz), 3.89 (s, 3H), 4.05 (s, 3H), 8.44 (s, 1H), 12.63 (s, 1H); ¹³C NMR (100 MHz, CDCl₃) δ13.88, 22.43, 24.44, 31.41, 38.19, 54.40, 60.57, 114.55, 130.525, 145.07, 160.81, 161.39, 206.02; HRMS (ESI) m/z calcd for C₁₃H₁₉NO₄ (M+Na⁺) 276.1206 found 276.1207.

2,3-dimethoxypyridin-4-yl diisopropylcarbamate (21).^{[31]} To a solution of 2,3-dimethoxypyridin-4-ol (9) (0.30 g, 1.93 mmol ) in toluene (10 mL) was added silver carbonate (0.80 g, 2.9 mmol) and diisopropylcarbamyl chloride (0.484 g, 2.9 mmol) and the reaction mixture was refluxed for 3 h. The suspension was allowed to cool and passed through celite® washing with MeOH, removal of the solvent in vacuo and purification by chromatography (DCM:Et₂O 96:4) provided the title compound as a colorless oil with yield (398 mg, 73%): ¹H NMR (400 MHz, CDCl₃) δ1.26 (d, 12H, J=6.8 Hz), 3.81 (s, 3H), 3.95 (s, 3H), 3.99 (m, 2H), 6.68 (d, 1H, J=5.6 Hz), 7.77 (d, 1H, J=5.6 Hz); ¹³CNMR (100 MHz, CDCl₃) δ20.2, 21.0, 46.6, 53.5, 60, 112.7, 135.7 140.4, 150.8, 152.1, 158.8.

5-bromo-2,3-dimethoxypyridin-4-yl diisopropylcarbamate (22).^{[31]} To a solution of 2,3-dimethoxypyridin-4-yl diisopropylcarbamate (21) (0.60 g, 2.12 mmol) in CCl₄ (20 mL) was added bromine (0.273 mL, 5.31 mmol) and the reaction mixture stirred at room temperature for 48 h, which was shielded from light with aluminum foil. To the reaction mixture was added a solution of saturated aqueous NaHCO₃ (5 mL) and Na₂S₂O₃ (5 mL) and extracted with ethyl acetate. The organic phase was washed with brine and dried over sodium sulfate, the solvent was removed in vacuo and the residue purified by column chromatography (DCM:Et₂O 98:2) to afford the title compound as a yellow solid (383 mg, 50%): ¹H NMR (400 MHz, CDCl₃) δ1.29 and 1.34 (2d, 12H, J=6.8 Hz), 3.86 (s, 3H), 3.97 (s, 3H), 4.01-4.04 (m, 2H), 7.99 (s, 1H); ¹³C NMR (100 MHz, CDCl₃) δ19.9, 20.8, 46.6, 46.9, 53.4, 58.9, 107.5, 137.1, 141.1, 148.3, 150.5, 157.5.

5-(1-hydroxyhexyl)-2,3-dimethoxypyridin-4-yl diisopropylcarbamate (23). To a solution of 5-bromo-2,3-dimethoxypyridin-4-yl diisopropylcarbamate (22) (0.12 g, 0.33 mmol) in THF (3 mL) at −78° C. was added ⁿBuLi (0.45 mL, of a 2.5 M solution in hexanes, 1.1 mmol) which was stirred for 1 minute. Hexanal (0.24 mL, 1.66 mmol) was added and the reaction mixture stirred for 1 h. The reaction was quenched by addition of ethanol and extracted with ethyl acetate, the organic washings were washed with brine and dried over sodium sulfate, the solvent was removed in vacuo and the residue purified by column chromatography (Hexane:EtOAc 4:1) to afforded the title compound as a colorless oil (104.7 mg, 83%): ¹H NMR (400 MHz, CDCl₃) δ0.86 (t, 3H, J=7.0 Hz), 1.1-1.5 (m, 18H), 1.70-2.00 (m, 2H), 3.82 (s, 3H), 4.00 (s, 3H), 3.98-4.12 (m, 2H), 4.69 (t, 1H, J=6.7 Hz), 7.97 (s, 1H); ¹³C NMR (100 MHz, CDCl₃) δ14.1, 20.4, 21.3, 22.6, 25.7, 31.6, 36.7, 47, 47.1, 53.8, 60.1, 60.3, 128.1, 135.6, 138.2, 148.9, 153.1, 158.

2,3-dimethoxy-5-(1-methoxyhexyl)pyridin-4-ol (24). 5-(1-hydroxyhexyl)-2,3-dimethoxypyridin-4-yl diisopropylcarbamate (23) (0.090 g, 0.23 mmol) was refluxed in KOH solution (5 M, in methanol, 10 mL) for 20 h. The solvent was removed in vacuo and diluted acetic acid added. The reaction mixture was neutralized by addition of saturated aqueous NaHCO₃, extracted with ethyl acetate. The organic washings were washed with brine and dried over sodium sulfate, the solvent was removed in vacco and purification by column chromatography afforded the title compound as a colorless oil (50%): ¹H NMR (400 MHz, CDCl₃) δ0.85 (t, 3H, J=7.0 Hz), 1.26 (m, 6H), 1.68-1.83 (m, 2H), 3.34 (s, 3H), 3.90 (s, 3H), 3.98 (s, 3H) 4.28 (t, 1H, J=6.7 Hz), 7.55 (s, 1H), 7.68 (s, 1H); ¹³C NMR (100 MHz, CDCl₃) δ13.9, 22.2, 25, 31.5, 35.7, 53.6, 57.1, 60.7, 80.7, 117.9, 130.4, 139.8, 154.8, 157.1; HRMS (ESI) m/z calcd for C₁₄H₂₃NO₄ (M+Na⁺) 292.1525 found 292.1517.

Complex II inhibition Assay. Mitochondria were isolated from fresh rat hearts by differential centrifugation in sucrose-based buffer as previously described.^{[11]} Complex II enzymatic activity was determined spectrophotometrically as the rate of succinate-driven, co-enzyme Q2-linked reduction of dichlorophenolindophenol (DCPIP). Mitochondria or sub-mitochondrial particles were incubated in phosphate buffer (pH 7.4) containing 40 μM DCPIP, 1 mM KCN, 10 μM rotenone, and 50 μM co-enzyme Q2. The rate of reduction of DCPIP to DCPIPH2 was followed at 600 nM (ε=21 mM⁻¹ cm⁻¹).^{[40]} The reaction was initiated by addition of succinate (10 mM), and varying amounts of inhibitors were used to determine an IC₅₀ value. At the end of each run thenoyltrifluoroacetone (1 mM) was added and the residual TTFA-insensitive rate subtracted.

Complex I inhibition Assay. Complex I (NADH ubiquinone-oxidoreductase) was measured spectrophotometrically (340 nm) in frozen/thawed mouse cardiac mitochondria, as the rotenone-sensitive oxidation of NADH (40 uM) in the presence of co-enzyme Q1 (10 uM), in phosphate buffer at pH 7.2, according to literature procedures.^{[41]}

Cytotoxicity Assays. DU-145, PC3, 22RV1 and HEK293 cell lines were purchased from ATCC and cultured according to the suppliers recommended protocol. To determine the cell growth inhibition ability of the synthesized compounds, cells were seeded at a density of 0.1×10⁶ cells/mL in 96 well plates containing 100 μL cell suspension per well. Stock solutions of the synthesized compounds were prepared in DMSO. Cells were treated at the indicated concentrations of test compounds, limiting the final DMSO concentration to less than 1%. After incubation at 37° C., 5% CO₂ for 48 hr, 20 μL of (3-(4,5-dimethylthiazol-2-yl)-5-(3-carboxymethoxyphenyl)-2-(4-sulfophenyl)-2H-tetrazolium) (MTS) reagent (CellTiter 96® AQueous One Solution Reagent) was added to each well and incubated at the above mentioned conditions for 3-4 hr. Plates were read at OD 490 nm on a plate reader and the viability of cells were plotted as percentage of controls.

Human PC3 prostate cancer cells were cultured in a microfluidic chip for 16 hours and the chip placed in a hypoxia chamber and pre-conditioned at <1% oxygen before treating the cells with various concentrations of synthesized compounds. Apoptosis was assayed using Annexin V and Sytox Green dye.^{[42]}

Assessment of Metabolic Parameters. Changes in oxygen consumption rate were measured using a XF24 Extracellular Flux Analyzer (Seahorse Bioscience, Billerica, MA). 22Rv1 cells were plated in XF24-well plates (40,000 cells/well) and allowed to adapt overnight. Treatments were performed with 16c at concentrations of 1, 10, 20, 30, 50 and 100 μg/mL and incubated for 48 hours. Mito Stress test was performed using oligomycin (10 μM), FCCP (5 μM) and Rotenone/Antimycin A (5 μM) based on the manufacturer's recommended protocol.

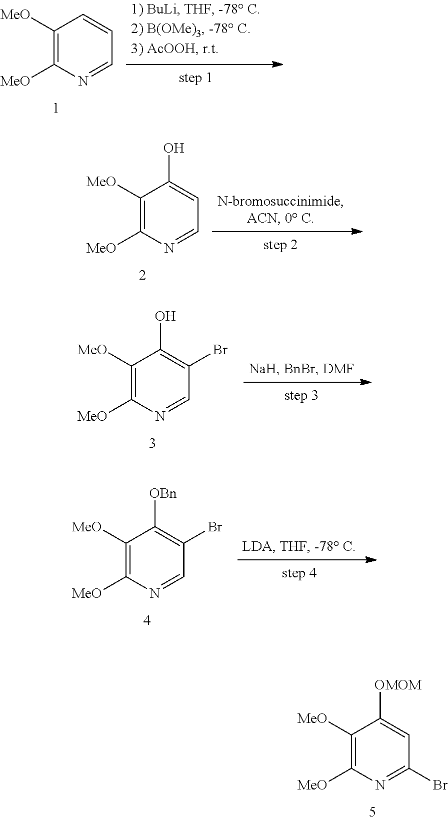

Step 1: 2,3-dimethoxypyridine (1, 10.0 g, 1.00 Eq, 71.8 mmol) was dissolved in THF (60.0 mL) at −78° C. under an N₂ atmosphere which was followed by the addition of nBuLi (2.5 M in hexane, 37.0 mL, 1.30 Eq, 93.4 mmol). The mixture was stirred for 1 h at 0° C. The reaction mixture was cooled to −78° C., trimethyl borate (16.0 mL, 2.00 Eq, 143 mmol) was added and the mixture was stirred at −78° C. for 2 h. A solution of peracetic acid (32 wt % in acetic acid) peracetic acid (9.55 mL, 2.00 Eq, 71.8 mmol) was added. The mixture was warmed to r.t. over 1 h. The reaction mixture was quenched by the addition of an aqueous solution of sodium thiosulfate. The mixture was extracted with ethyl acetate (50 mL×3). The organic phases were combined, washed with brine, and dried over sodium sulfate, filtered, concentrated, and purified by flash column chromatography (SiO_{2,} 80 g cartridge, gradient from 5% to 20% ethyl acetate in hexane). 2,3-dimethoxypyridin-4-ol (2, 4.10 g, colorless oil, 37%) was obtained. R_{f}=0.30 (hexane/ethyl acetate=4/1).

Step 2: 2,3-dimethoxypyridin-4-ol (2, 4.10 g, 1.00 Eq, 26.0 mmol) was dissolved in Acetonitrile (30 mL) at r.t., which was followed by the addition of N-bromosuccinimide (NBS) (5.20 g, 1.10 Eq, 29.0 mmol). The reaction mixture was quenched by addition of H₂O, diluted with ethyl acetate (30.0 mL), and washed with H₂O and brine. The organic phase dried over Na₂SO₄, filtered, and concentrated under vacuum. The residue was mixed with silica gel and purified by column chromatography (SiO_{2,} 40 g cartridge, gradient from 5% to 20% ethyl acetate in hexane). 5-bromo-2,3-dimethoxypyridin-4-ol (3, 3.60 g, colorless oil, 58%) was obtained. R_{f}=0.60 (hexane/ethyl acetate=4/1).

Step 3: 5-bromo-2,3-dimethoxypyridin-4-ol (3, 3.60 g, 1.00 Eq, 15.0 mmol) was dissolved in DMF (10.0 mL) at 0° C. NaH (0.74 g, 2.00 Eq, 31.0 mmol) was added to the reaction mixture. 10 min later, MOM-Cl (2.30 mL, 2.00 Eq, 31.0 mmol) was added to the reaction mixture. The reaction mixture was allowed to warm to room temperature and stirred for another 1 h. The reaction mixture was quenched by addition of H₂O, diluted with ethyl aetate (30 mL), and washed with H₂O and brine. The organic phase dried over Na₂SO₄, filtered, and concentrated under vacuum. The residue was mixed with silica gel and purified by column chromatography (SiO_{2,} 25 g cartridge, gradient from 5% to 10% ethyl acetate in hexane). 5-bromo-2,3-dimethoxy-4-(methoxymethoxy)pyridine (4, 4.30 g, colorless oil, 90%) was obtained. R_{f}=0.65 (hexane/ethyl acetate=4/1).

Step 4: To a solution of diisopropylamine (6.93 mL, 3.00 Eq, 49.1 mmol) in THF (10.0 mL), nBuLi (2.5 M in hexane, 19.6 mL, 3.00 Eq, 49.1 mmol) was added at −78° C. The reaction mixture was stirred at 0° C. for 30 min, which was cooled down to −78° C. and added to a solution of 5-bromo-2,3-dimethoxy-4-(methoxymethoxy)pyridine (4, 4.30 g, 1.00 Eq, 16.4 mmol) in THF (10.0 mL) at −78° C. An excess of MeOH (4.0 mL) was added and the mixture was warmed up to 23° C., which was treated with a saturated aqueous solution of NH₄Cl (5.0 mL) and then extracted with ethyl acetate. The reaction mixture was quenched by the addition of NH₄Cl (aq. sat., 10.0 mL), extracted with ethyl acetate (30 mL), and washed with H₂O and brine. The organic phase was dried over Na₂SO₄,filtered, and concentrated under vacuum. The residue was mixed with silica gel and purified by column chromatography (SiO_{2,} 25 g cartridge, gradient from 0.5% to 2% ethyl acetate in hexane). 6-bromo-2,3-dimethoxy-4-(methoxymethoxy)pyridine (5, 1.60 g, white solid, 35%) was obtained. R_{f}=0.40 (hexane/ethyl acetate=4/1).

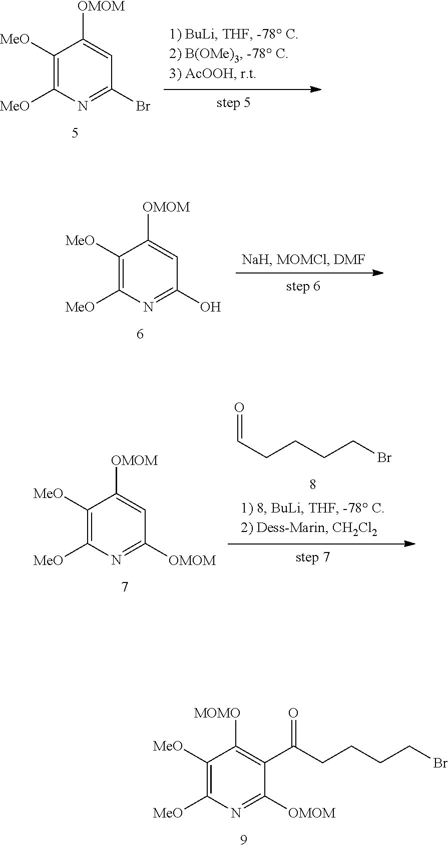

Step 5: 6-bromo-2,3-dimethoxy-4-(methoxymethoxy)pyridine (5, 1.60 g, 1.00 Eq, 5.75 mmol) was dissolved in THF (1.0 mL) at −78° C. under an N2 atmosphere which was followed by quick addition of nBuLi (2.5 M in hexane, 4.60 mL, 2.00 Eq, 11.5 mmol). 1 min later, trimethyl borate (1.92 mL, 3.00 Eq, 17.3 mmol) was added and the mixture was stirred at −78° C. for 2 h. A solution of peracetic acid (32 wt % in acetic acid, 1.15 mL, 3.00 Eq, 17.3 mmol) was added. The mixture was warmed to 0° C. The reaction mixture was quenched with Na₂S₂O₂ (Sat.), extracted with ethyl acetate (30 mL×3). The combined organic phases were dried over sodium sulfate, filtered and concentrated under vacuum. The residue was purification by flash column chromatography (SiO_{2,} 25 g cartridge, gradient from 0% to 2% MeOH in CH₂Cl₂). 5,6-dimethoxy-4-(methoxymethoxy)pyridin-2-ol (6, 1.05 g, colorless oil, 85%) was obtained. R_{f}=0.30 (CH₂Cl₂/MeOH=50/1).

Step 6: 5,6-dimethoxy-4-(methoxymethoxy)pyridin-2-ol (6, 1.05 g, 1.00 Eq, 4.88 mmol) was dissolved in DMF (10 mL) at 0° C. NaH (0.23 g, 2.00 Eq, 9.76 mmol) was added to the reaction mixture. 10 min later, MOM-Cl (741 μL, 2.00 Eq, 9.76 mmol) was added to the reaction mixture. The reaction mixture was allowed to warm to room temperature and stirred for another 1 h. The reaction mixture was quenched by addition of H₂O (20 mL), diluted with ethyl acetate (30 mL), and washed with H₂O and brine. The organic phase dried over Na₂SO₄, filtered, and concentrated under vacuum. The residue was mixed with silica gel and purified by column chromatography (SiO_{2,} 12 g cartridge, gradient from 5% to 10% ethyl acetate in hexane). 2,3-dimethoxy-4,6-bis(methoxymethoxy)pyridine (7, 0.800 g, colorless oil, 63%) was obtained. R_{f=}0.50 (hexane/ethyl acetate=4/1).

Step 7: 2,3-dimethoxy-4,6-bis(methoxymethoxy)pyridine (7, 100 mg, 1.00 Eq, 386 μmol) was dissolved in THF (10 mL) at −78° C. nBuLi (1.6 M in hexane, 312 μmL, 1.30 Eq, 501 μmol) was added to the reaction mixture. The reaction mixture was warmed up to 0° C. and stirred for another 1 h followed by 5-bromopentanal (8, 95.5 mg, 1.5 Eq, 579 μmol) was added to the reaction mixture. 2 h later, the reaction mixture was quenched by addition of H₂O, diluted with ethyl acetate (30 mL), and washed with H₂O and brine. The skilled artisan will recognize that the 5-bromopentanal can be substituted with molecules that have different chain lengths (e.g., C₁ to C₁₅) and side groups. The organic phase dried over Na₂SO₄,filtered, and concentrated under vacuum. The residue was used directly for Dess Martin oxidation. The residue was dissolved in CH₂Cl₂ (10 mL) at 0° C. Dess-Martin periodinane (246 mg, 1.50 Eq, 580 μmol) was added to the reaction mixture. The reaction mixture was warmed up to room temperature and stirred for another 1 h. The reaction mixture was quenched by addition of H₂O, diluted with ethyl acetate (30 mL), and washed with H₂O and brine. The organic phase dried over Na₂SO₄, filtered, and concentrated under vacuum. The residue was purified by column chromatography (SiO_{2,} 12 g cartridge, gradient from 2% to 7% ethyl acetate in hexane). 5-bromo-1-(5,6-dimethoxy-2,4-bis(methoxymethoxy)pyridin-3-yl)pentan-1-one (9, 60 mg, colorless oil, 37%) was obtained. R_{f}=0.30 (hexane/ethyl acetate=4/1).

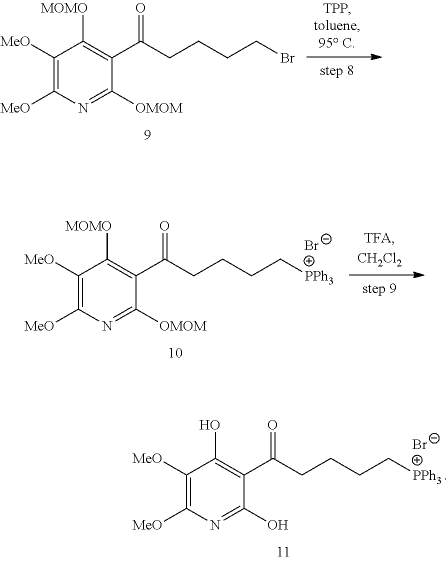

Step 8: 5-bromo-1-(5 ,6-dimethoxy-2,4-bis(methoxymethoxy)pyridin-3-yl)pentan-1-one (9, 180 mg, 1.00 Eq, 426 μmol) was dissolved in toluene (5 mL) at 95° C. triphenylphosphine (559 mg, 5.00 Eq, 2.13 mmol) was added to the reaction mixture. The reaction mixture was stirred overnight at 95° C. The reaction mixture was concentrated under vacuum. The residue was purified by column chromatography (SiO_{2,} 4 g cartridge, gradient from 0.6% to 6% MeOH in CH₂Cl₂). (5-(5,6-dimethoxy-2,4-bis(methoxymethoxy)pyridin-3-yl)-5-oxopentyl)-triphenyl-phosphonium, Bromide (10, 70 mg, 0.10 mmol, 24%) mixture of product and de MOM product. R_{f}=0.40 (CH₂Cl₂/MeOH=10/1).

Step 9: (5-(5,6-dimethoxy-2,4-bis(methoxymethoxy)pyridin-3-yl)-5-oxopentyl) triphenyl phos-phonium, Bromide (10, 70 mg, 1.0 Eq, 0.10 mmol) was dissolved in CH₂Cl₂ (5 mL) at 25° C. TFA (0.39 mL, 50 Eq, 5.1 mmol) was added to the reaction mixture. The reaction mixture was stirred over night at 25° C. The reaction mixture was concentrated under vacuum. The residue was purified by column chromatography (SiO_{2,} 4 g cartridge, gradient from 1% to 8% MeOH in CH₂Cl₂). (5-(2,4-dihydroxy-5,6-dimethoxypyridin-3-yl)-5-oxopentyl)triphenylphosphonium, Bromide (11, 50 mg, colorless oil, 84%) was obtained. R_{f}=0.35 (CH₂Cl₂/MeOH=10/1).

¹H NMR (CDCl_{3,} 600 MHz): δ7.83-7.70 (m, 15H), 4.06 (s, 3H), 3.79 (s, 3H), 3.52-3.50 (m, 2H), 3.12 (t, J=7.5 Hz, 2H), 1.99-1.95 (m, 2H), 1.78-1.73 (m, 2H).

¹³C NMR (CDCl_{3,} 150 MHz): δ204.47, 161.22, 158.45, 134.73 (P-C, J=2.90 Hz), 133.03 (P-C, J=9.87 Hz), 130.12 (P-C, J=5.76 Hz), 122.94, 117.79 (P-C, J=85.59 Hz), 99.64, 60.59, 40.93, 25.17 (P-C, J=550.94 Hz), 21.39 (P-C, J=51.12 Hz).

HRMS: calcd: C₃₀H₃₁NO₅P⁺ [M+H]⁺: 516.1934; found: 516.1949.

HPLC: 97.3%.

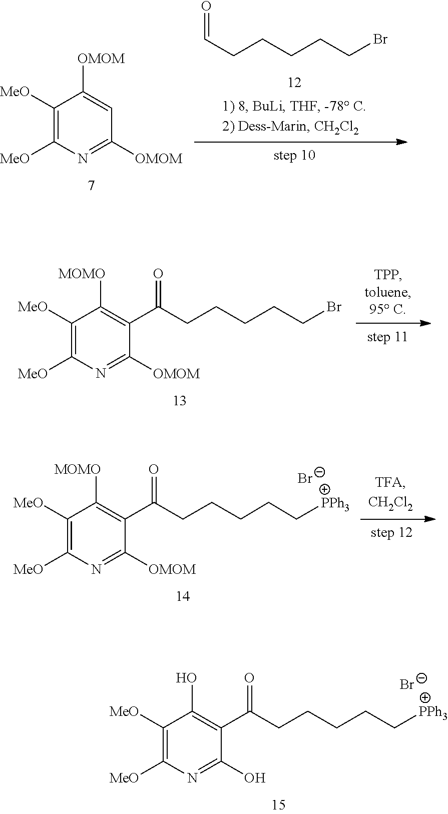

Step 10: 2,3-dimethoxy-4,6-bis(methoxymethoxy)pyridine (7, 190 mg, 1.00 Eq, 733 μmol) was dissolved in THF (10 mL) at −78° C. nBuLi (1.6 M in hexane, 1.50 Eq, 1.10 mmol) was added to the reaction mixture. The reaction mixture was warmed up to 0° C. and stirred for another 1 h followed by 6-bromohexanal (12, 197 mg, 1.50 Eq, 1.10 mmol) was added to the reaction mixture. 2 h later, the reaction mixture was quenched by addition of H₂O, diluted with ethyl acetate (30 mL), and washed with H₂O and brine. The organic phase dried over Na₂SO₄, filtered, and concentrated under vacuum. The residue was used directly for Dess Martin oxidation. The residue was dissolved in CH₂Cl₂ (10 mL) at 0° C. Dess-Martin periodinane (466 mg, 1.50 Eq, 1.19 mmol) was added to the reaction mixture. The reaction mixture was warmed up to room temperature and stirred for another 1 h. The reaction mixture was quenched by addition of H₂O, diluted with ethyl acetate (30 mL), and washed with H₂O and brine. The organic phase dried over Na₂SO₄, filtered, and concentrated under vacuum. The residue was purified by column chromatography (SiO_{2,} 12 g cartridge, gradient from 2% to 10% ethyl acetate in hexane). 6-bromo-1-(5,6-dimethoxy-2,4-bis(methoxymethoxy)pyridin-3-yl)hexan-1-one (13, 150 mg, colorless oil, 47%) was obtained. R_{f}=0.30 (hexane/ethyl acetate=4/1).

Step 11: 6-bromo-1-(5,6-dimethoxy-2,4-bis(methoxymethoxy)pyridin-3-yl)hexan-1-one (13, 150 mg, 1.00 Eq, 344 μmol) was dissolved in toluene (5 mL) at 95° C. Triphenylphosphine (451 mg, 5 Eq, 1.72 mmol) was added to the reaction mixture. The reaction mixture was stirred overnight at 95° C. The reaction mixture was concentrated under vacuum. The residue was purified by column chromatography (SiO_{2,} 4 g cartridge, gradient from 0.6% to 6% MeOH in CH₂Cl₂). (6-(5,6-dimethoxy-2,4-bis(methoxymethoxy)pyridin-3-yl)-6-oxohexyl)triphenylphosphonium, Bromide (14, 144 mg, 60%) mixture of product and de MOM product. R_{f}=0.40 (CH₂Cl₂/MeOH=10/1).

Step 12: (6-(5,6-dimethoxy-2,4-bis(methoxymethoxy)pyridin-3-yl)-6-oxohexyl)triphenyl-phos-phonium, Bromide (14, 144 mg, 1.00 Eq, 206 μmol) was dissolved in CH₂Cl₂ (5 mL) at 25° C. TFA (794 μL, 50 Eq, 10.3 mmol) was added to the reaction mixture. The reaction mixture was stirred overnight at 25° C. The reaction mixture was concentrated under vacuum. The residue was purified by column chromatography (SiO_{2,} 4 g cartridge, gradient from 1% to 8% MeOH in CH₂Cl₂). (6-(2,4-dihydroxy-5,6-dimethoxypyridin-3-yl)-6-oxohexyl)triphenylphosphonium, Bromide (15, 60 mg, colorless oil, 48%) was obtained. R_{f}=0.35 (CH₂Cl₂/MeOH=10/1).

¹NMR (CDCl_{3,} 600 MHz): δ7.83-7.69 (m, 15H), 4.07 (s, 3H), 3.77 (s, 3H), 3.43-3.41 (m, 2H), 3.01 (t, J=6.18 Hz, 2H), 1.68 (m, 6H).

¹³C NMR (CDCl_{3,} 150 MHz): δ205.71, 161.19, 157.65, 134.70 (P-C, J=2.94 Hz), 133.00 (P-C, J=9.86 Hz), 130.11 (P-C, J=12.44 Hz), 122.38, 117.87 (P-C, J=85.58 Hz), 99.87, 60.62, 41.72, 29.48 (P-C, J=15.83 Hz), 23.62 (P-C, J=34.38 Hz), 21.73 (P-C, J=39.20 Hz).

HRMS: calcd: C₃₀H₃₁NO₅P⁺ [M+H]⁺: 530.2091; found: 530.2116.

HPLC: 98.9%.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph δof 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

### [1] F. Sun, X. Huo, Y. Zhai, A. Wang, J. Xu, D. Su, M. Bartlam, Z. Rao, Cell 2005, 121, 1043-1057.

[2] L. Ernster, G. Dallner, Biochim. Biophys. Acta. 1995, 1271, 195-204.
[3] K. Kluckova, A. Bezawork-Geleta, J. Rohlena, L. Dong, J. Neuzil, Biochim. Biophys. Acta. 2013, 1827, 552-564.

### [4] M. A. Selak, S. M. Armour, E. D. MacKenzie, H. Boulahbel, D. G. Watson, K. D. Mansfield, Y. Pan, M. C. Simon, C. B. Thompson, E. Gottlieb, Cancer Cell 2005, 7, 77-85.

[5] K. Kluckova, M. Sticha, J. Cerny, T. Mracek, L. Dong, Z. Drahota, E. Gottlieb, J. Neuzil, J. Rohlena, Cell Death Dis. 2015, 6, e1749.
[6] M. Peczkowska, A. Cascon, A. Prejbisz, A. Kubaszek, B. J. Cwikla, M. Furmanek, Z. Erlic, C. Eng, A. Januszewicz, H. P. Neumann, Nat. Clin. Pract. Endocrinol. Metab. 2008, 4, 111-115.

### [7] E. Tomitsuka, K. Kita, H. Esumi, J. Biochem. 2012, 152, 171-183.

### [8] Y. Chen, E. McMillan-Ward, J. Kong, S. J. Israels, S. B. Gibson, J. Cell Sci. 2007, 120, 4155-4166.

[9] S. Fulda, Int. J. Cancer 2009, 124, 511-515.
[10] J. Neuzil, J. C. Dyason, R. Freeman, L. F. Dong, L. Prochazka, X. F. Wang, I. Scheffler, S. J. Ralph, J. Bioenerg. Biomembr. 2007, 39, 65-72.
[11] A. P. Wojtovich, P. S. Brookes, Biochim. Biophys. Acta. 2008, 1777, 882-889.
[12] J. Gruber, K. Staniek, C. Krewenka, R. Moldzio, A. Patel, S. Bohmdorfer, T. Rosenau, L. Gille, Bioorg. Med. Chem. 2014, 22, 684-691.

### [13] J. Neuzil, L. F. Dong, J. Rohlena, J. Truksa, S. J. Ralph, Mitochondrion 2013, 13, 199-208.

[14] L. F. Dong, V. J. Jameson, D. Tilly, J. Cerny, E. Mandavian, A. Marin-Hernandez, L. Hernandez-Esquivel, S. Rodriguez-Enriquez, J. Stursa, P. K. Witting, B. Stantic, J. Rohlena, J. Truksa, K. Kluckova, J. C. Dyason, M. Ledvina, B. A. Salvatore, R. Moreno-Sanchez, M. J. Coster, S. J. Ralph, R. A. Smith, J. Neuzil, J. Biol. Chem. 2011, 286, 3717-3728.

### [15] S. Nadanaciva, A. Bernal, R. Aggeler, R. Capaldi, Y. Will, Toxicol. in Vitro 2007, 21, 902-911.

[16] P. P. Dzeja, P. Bast, C. Ozcan, A. Valverde, E. L. Holmuhamedov, D. G. Van Wylen, A. Terzic, Am. J. Physiol. Heart Circ. Physiol. 2003, 284, H1048-1056.
[17] S. J. Ralph, R. Moreno-Sanchez, J. Neuzil, S. Rodriguez-Enriquez, Pharm. Res. 2011, 28, 2695-2730.
[18] E. T. Chouchani, V. R. Pell, E. Gaude, D. Aksentijevic, S. Y. Sundier, E. L. Robb, A. Logan, S. M. Nadtochiy, E. N. Ord, A. C. Smith, F. Eyassu, R. Shirley, C. H. Hu, A. J. Dare, A. M. James, S. Rogatti, R. C. Hartley, S. Eaton, A. S. Costa, P. S. Brookes, S. M. Davidson, M. R. Duchen, K. Saeb-Parsy, M. J. Shattock, A. J. Robinson, L. M. Work, C. Frezza, T. Krieg, M. P. Murphy, Nature 2014, 515, 431-435.
[19] L. Valls-Lacalle, I. Barba, E. Miro-Casas, J. J. Alburquerque-Bejar, M. Ruiz-Meana, M. Fuertes-Agudo, A. Rodriguez-Sinovas, D. Garcia-Dorado, Cardiovasc. Res. 2016, 109, 374-384.
[20] A. P. Wojtovich, P. S. Brookes, Basic Res. Cardiol. 2009, 104, 121-129.
[21] A. S. Hoekstra, J. P. Bayley, Biochim. Biophys. Acta. 2013, 1827, 543-551.
[22] G. Touati, F. Poggi-Travert, H. Ogier de Baulny, J. Rahier, F. Brunelle, C. Nihoul-Fekete, P. Czernichow, J. M. Saudubray, Eur. J. Pediatr. 1998, 157, 628-633.
[23] H. Miyadera, K. Shiomi, H. Ui, Y. Yamaguchi, R. Masuma, H. Tomoda, H. Miyoshi, A. Osanai, K. Kita, S. Omura, Proc. Nat. Acad. Sci. U. S. A. 2003, 100, 473-477.
[24] I. Siebels, S. Drose, Biochim. Biophys. Acta. 2013, 1827, 1156-1164.

### [25] M. Kawada, I. Momose, T. Someno, G. Tsujiuchi, D. Ikeda, J. Antibiot. 2009, 62, 243-246.

[26] M. Kawada, H. Inoue, S. Ohba, T. Masuda, I. Momose, D. Ikeda, Int. J. Cancer 2010, 126, 810-818.
[27] T. P. Selby, K. A. Hughes, J. J. Rauh, W. S. Hanna, Bioorg. Med. Chem. Lett. 2010, 20, 1665-1668.
[28] R. Horsefield, V. Yankovskaya, G. Sexton, W. Whittingham, K. Shiomi, S. Omura, B. Byrne, G. Cecchini, S. Iwata, J. Biol. Chem. 2006, 281, 7309-7316.

### [29] M. Ohtawa, S. Ogihara, K. Sugiyama, K. Shiomi, Y. Harigaya, T. Nagamitsu, S. Omura, J. Antibiot. 2009, 62, 289-294.

[30] S. Krautwald, C. Nilewski, M. Mori, K. Shiomi, S. Omura, E. M. Carreira, Angew. Chem. 2016, 55, 4049-4053.
[31] F. Trecourt, M. Mallet, O. Mongin, G. Queguiner, J. Org. Chem. 1994, 59, 6173-6178.
[32] P. C. Trippier, C. McGuigan, J. Balzarini, Antivir. Chem. Chemother. 2010, 20, 249-257.
[33] P. D. Leeson, B. Springthorpe, Nat. Rev. Drug Discov. 2007, 6, 881-890.
[34] K. L. Horton, K. M. Stewart, S. B. Fonseca, Q. Guo, S. O. Kelley, Chem. Biol. 2008, 15, 375-382.

### [35] I. Reulecke, G. Lange, J. Albrecht, R. Klein, M. Rarey, ChemMedChem 2008, 3, 885-897.

### [36] Y. Li, S. C. Chan, L. J. Brand, T. H. Hwang, K. A. Silverstein, S. M. Dehm, Cancer Res. 2013, 73, 483-489.

[37] C. Wenzel, B. Riefke, S. Grundemann, A. Krebs, S. Christian, F. Prinz, M. Osterland, S. Golfier, S. Rase, N. Ansari, M. Esner, M. Bickle, F. Pampaloni, C. Mattheyer, E. H. Stelzer, K. Parczyk, S. Prechtl, P. Steigemann, Exp. Cell. Res. 2014, 323, 131-143.
[38] T. Germain, M. Ansari, D. Pappas, Anal. Chim. Acta 2016, 936, 179-184.

### [39] P. J. Maxwell, R. Gallagher, A. Seaton, C. Wilson, P. Scullin, J. Pettigrew, I. J. Stratford, K. J. Williams, P. G. Johnston, D. J. Waugh, Oncogene 2007, 26, 7333-7345.

[40] C. I. Ragan, M. T. Wilson, V. M. Darley-Usmar, P. N. Lowe, Sub-fractionation of mitochondria and isolation of the roteins of oxidative phosphorylation. Chapter 4 In: Mitochondria, a Practical Approach, IRL Press, Oxford, UK, 1988.

### [41] D. M. Kirby, D. R. Thurburn, D. M. Turnbull, Methods Cell Biol. 2007, 80, 93-119.

### [42] G. Khanal, S. Hiemstra, D. Pappas, Analyst 2014, 139, 3274-3280.

What is claimed is:

1. A compound comprising:
an Atpenin 5 derivative of Formula: 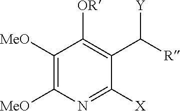
wherein R′ is selected from H, methoxy, methoxymethyl, glycosyl, phosphoryl, alkylamine, polyethylene glycol (PEG), or deuterated; X is selected from OH, methoxy, methoxymethyl, O-methoxymethyl, carbonyl, or alcohol; Y is O; R″ is selected from H, pentyl, hexyl, heptyl, octyl, nonyl, decyl, or dodecyl, that are saturated or unsaturated, a PEG, a triphenylphosphate group; optionally one or both MeO groups are deuterated; and N is N-oxide or N-alkyl.

2. The compound of claim 1, wherein the compound comprises: 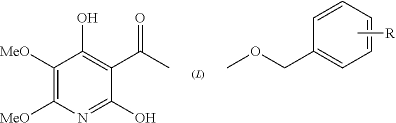
L is a linker that is C₁-C₁₅ or polyethyleneglycol 1-7 units, wherein L can be odd or even;
R=2,3 or 4 mono, di, tri substitution with halogen electron-donating group or electron-withdrawing groups; and
Ether Oxygen may be NH, S, or C.

3. The compound of claim 2, wherein the compound comprises: 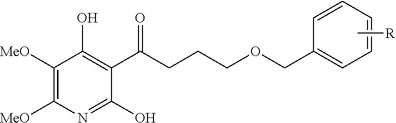
R=2,3 or 4 mono, di, tri substitution with halogen electron-donating group or electron-withdrawing groups; and
Ether Oxygen may be NH, S, or C.

4. The compound of claim 1, wherein the compound comprises:

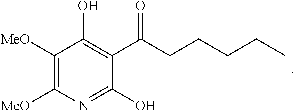 [SMILES: CCCCCC(=O)c1c(O)nc(OC)c(OC)c1O]

5. The compound of claim 1, wherein the compound comprises:

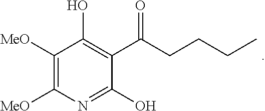 [SMILES: CCCCC(=O)c1c(O)nc(OC)c(OC)c1O]

6. The compound of claim 1, wherein the compound comprises:

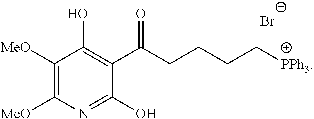

7. The compound of claim 1, wherein the compound comprises:

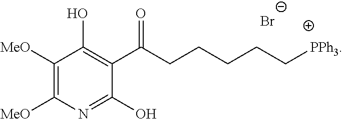

8. The compound of claim 1, wherein the compound comprises at least one of: 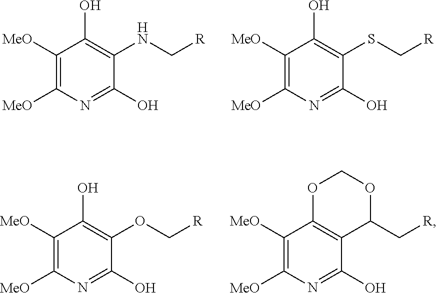
wherein NH is optionally alkylated, sulphur oxidation state will vary S(O) or S(O₂), and R is R′ or R″.

9. The compound of claim 1, wherein the compound comprises: 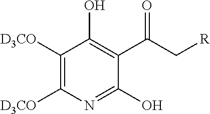
wherein NH is optionally alkylated, sulphur oxidation state will vary S(O) or S(O₂), and R is R′ or R″.

10. The compound of claim 1, wherein the compound is adapted for oral, intravenous, subcutaneous, parenteral, enteral, transcutaneous, transdermal, or rectal administration.

11. The compound of claim 1, further comprising one or more pharmaceutically acceptable carriers, excipients, buffers, or salts.

12. A compound of Formula: 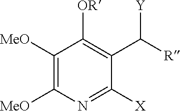
wherein R′ is selected from H, methoxy, methoxymethyl, glycosyl, phosphoryl, alkylamine, polyethylene glycol (PEG), or deuterated; X is selected from OH, methoxy, methoxymethyl, O-methoxymethyl, carbonyl, or alcohol; Y is O; R″ is selected from H, pentyl, hexyl, heptyl, octyl, nonyl, decyl, or dodecyl, that are saturated or unsaturated, PEG, or triphenylphosphate group; optionally one or both MeO groups are deuterated; and N is N-oxide or N-alkyl.

13. The compound of claim 12, wherein the compound comprises: 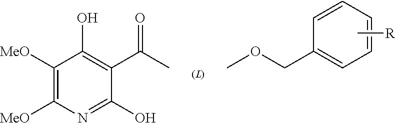
L is a linker that is C₁-C₁₅ or polyethyleneglycol 1-7 units, wherein L can be odd or even;
R=2,3 or 4 mono, di, tri substitution with halogen electron-donating group or electron-withdrawing groups; and
Ether Oxygen may be NH, S, or C.

14. The compound of claim 13, wherein the compound comprises: 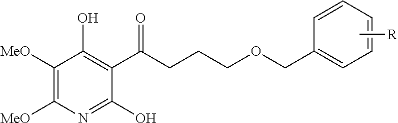
R=2,3 or 4 mono, di, tri substitution with halogen electron-donating group or electron-withdrawing groups; and
Ether Oxygen may be NH, S, or C.

15. The compound of claim 12, wherein the compound comprises:

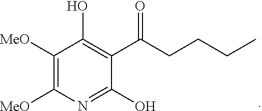 [SMILES: CCCCC(=O)c1c(O)nc(OC)c(OC)c1O]

16. The compound of claim 12, wherein the compound comprises:

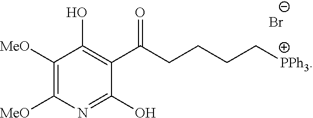

17. The compound of claim 12, wherein the compound comprises:

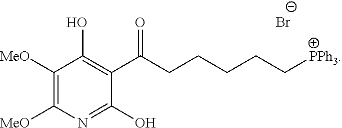

18. The compound of claim 12, wherein the compound comprises at least one of: 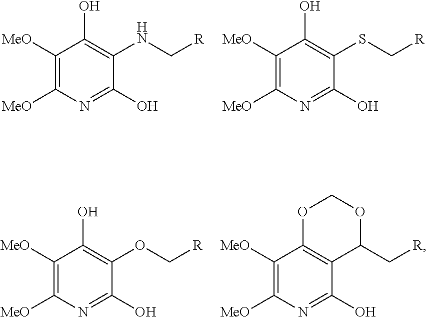
wherein NH is optionally alkylated, sulphur oxidation state will vary S(O) or S(O₂), and R is R′ or R″.

19. The compound of claim 12, wherein the compound comprises: 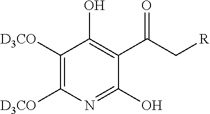
wherein NH is optionally alkylated, sulphur oxidation state will vary S(O) or S(O₂), and R is R′ or R″.

20. The compound of claim 12, wherein the composition is adapted for oral, intravenous, subcutaneous, parenteral, enteral, transcutaneous, transdermal, or rectal administration.

21. The compound of claim 12, further comprising adding one or more pharmaceutically acceptable carriers, excipients, buffers, or salts to the composition.

22. A method of treating a cancer cell comprising:
an effective amount of a composition comprising an Atpenin 5 derivative of Formula: 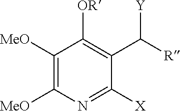
wherein R′ is selected from H, methoxy, methoxymethyl, glycosyl, phosphoryl, alkylamine, polyethylene glycol (PEG), or deuterated; X is selected from OH, methoxy, methoxymethyl, O-methoxymethyl, carbonyl, or alcohol; Y is O; R″ is selected from H, pentyl, hexyl, heptyl, octyl, nonyl, decyl, or dodecyl, that are saturated or unsaturated, PEG or triphenylphosphate group; optionally one or both MeO groups are deuterated; N is N-oxide or N-alkyl; and
one or more pharmaceutically acceptable carriers, excipients, buffers, or salts.

23. The method of claim 22, wherein the compound comprises: 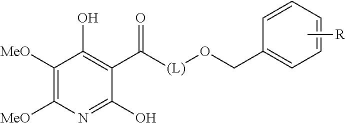
L is a linker that is C₁-C₁₅ or polyethyleneglycol 1-7 units, wherein L can be odd or even;
R=2,3 or 4 mono, di, tri substitution with halogen electron-donating group or electron-withdrawing groups; and
Ether Oxygen may be NH, S, or C.

24. The method of claim 23, wherein the compound comprises: 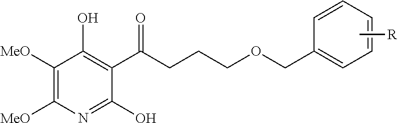
R=2,3 or 4 mono, di, tri substitution with halogen electron-donating group or electron-withdrawing groups; and
Ether Oxygen may be NH, S, or C.

25. The method of claim 22, wherein the compound comprises:

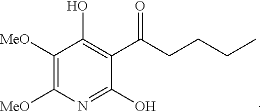 [SMILES: CCCCC(=O)c1c(O)nc(OC)c(OC)c1O]

26. The method of claim 22, wherein the compound comprises:

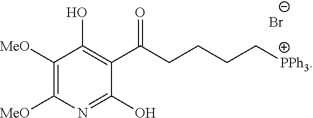

27. The method of claim 22, wherein the compound comprises:

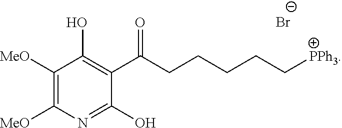

28. The method of claim 22, wherein the composition comprises at least one of: 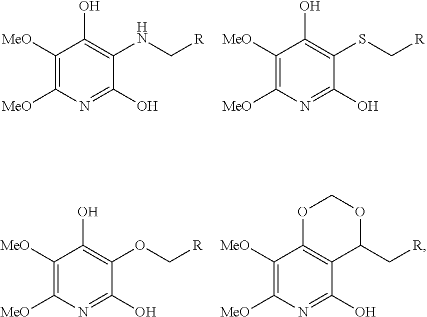
wherein NH is optionally alkylated, sulphur oxidation state will vary S(O) or S(O₂), and R is R′ or R″.

29. The method of claim 22, wherein the composition comprises: 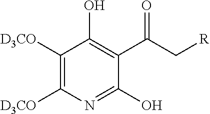
wherein NH is optionally alkylated, sulphur oxidation state will vary S(O) or S(O₂), and R is R′ or R″.

30. The method claim 22, wherein the composition is adapted for oral, intravenous, subcutaneous, parenteral, enteral, transcutaneous, transdermal, or rectal administration.

31. A method of making hydrocarbon side chain derivatives of Atpenin A5 comprising: 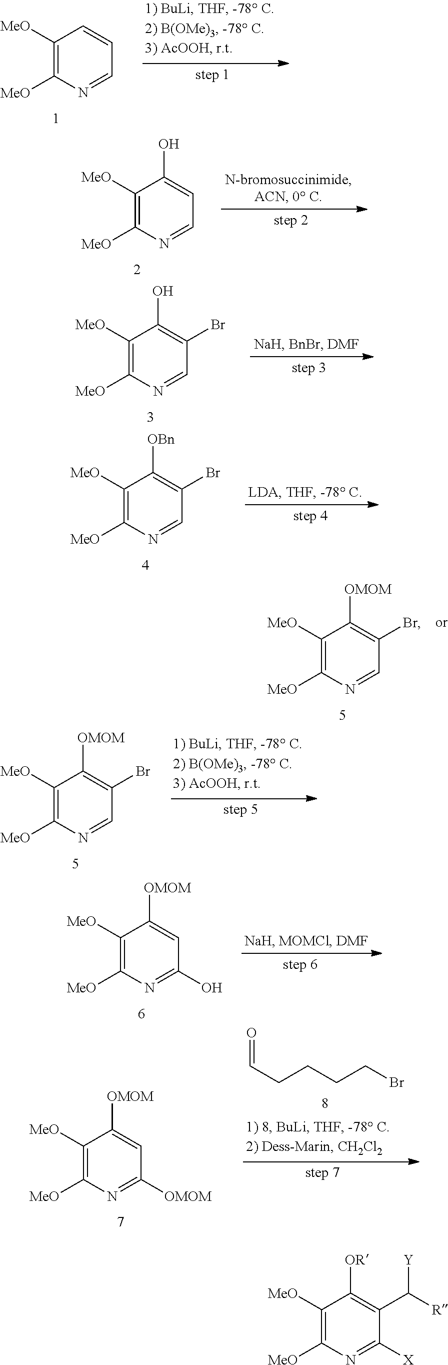
wherein R′ is selected from H, methoxy, methoxymethyl, glycosyl, phosphoryl, alkylamine, polyethylene glycol (PEG), or deuterated; X is selected from OH, methoxy, methoxymethyl, O-methoxymethyl, carbonyl, or alcohol; Y is O; and R″ is selected from H, butyl, pentyl, hexyl, heptyl, octyl, nonyl, decyl, or dodecyl, that are saturated or unsaturated, PEG, or triphenylphosphate; optionally one or both MeO groups are deuterated; and N is N-oxide or N-alkyl.

32. The method of claim 31, wherein the method further comprises:

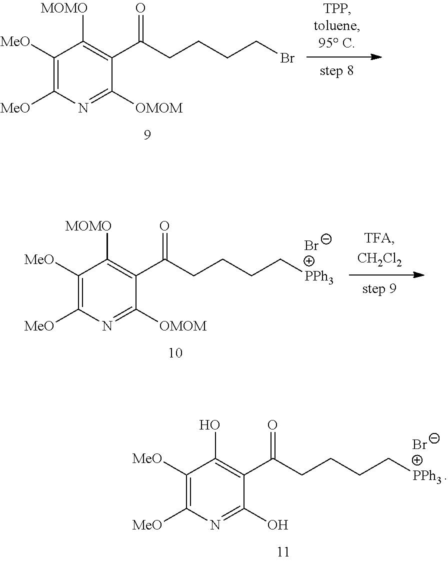

33. A method of treating chronic obstructive pulmonary disease comprising:
an effective amount of a composition comprising an Atpenin 5 derivative of Formula: 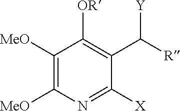
wherein R′ is selected from H, methoxy, methoxymethyl, glycosyl, phosphoryl, alkylamine, polyethylene glycol (PEG), or deuterated; X is selected from OH, methoxy, methoxymethyl, O-methoxymethyl, carbonyl, or alcohol; Y is O; R″ is selected from H, pentyl, hexyl, heptyl, octyl, nonyl, decyl, or dodecyl, that are saturated or unsaturated, PEG or triphenylphosphate group; optionally one or both MeO groups are deuterated; N is N-oxide or N-alkyl; and
one or more pharmaceutically acceptable carriers, excipients, buffers, or salts.
